(19) United States
(12) Patent Application Publication
(10) Pub. No.: US 2024/0059787 A1
(43) Pub. Date: Feb. 22, 2024

(54) HERV-K Antibody Therapeutics

(71) Applicants: Feng Wang-Johanning, Sunnyvale, CA (US); Gary Johanning, Sunnyvale, CA (US)

(72) Inventors: Feng Wang-Johanning, Sunnyvale, CA (US); Gary Johanning, Sunnyvale, CA (US)

(73) Assignee: SunnyBay Bio Tech, Inc., Fremont, CA (US)

(21) Appl. No.: 18/245,879

(22) PCT Filed: Sep. 18, 2021
(86) PCT No.: PCT/US2021/071505

§ 371 Date: Mar. 17, 2023

(51) Int. Cl. C07K 16/30; A61K 39/00; A61P 35/00; C07K 16/28

(52) U.S. Cl. CPC C07K 16/30; A61K 39/4611; A61K 39/4631; A61K 39/4644; A61K 39/464838; A61P 35/00; C07K 16/2809; C07K 16/2815; A61K 2039/505; C07K 2317/24; C07K 2317/31; C07K 2317/622; C07K 2317/92

(57) ABSTRACT

The invention provides therapeutic humanized anti-HERV-K antibodies, CAR, or a fusion thereof consisting of a hispecific T ceil engager (BiTE) FOR CD3 and CDS, a DNA-encoded BiTE (DBiTE), or an antibody-drug conjugate (ADC). The invention also relates to peptides, proteins, nucleic acids, and cells for use in immunotherapeutic methods. In particular, the invention relates to the immunotherapy of cancer peptides bound to molecules of the MHC, or peptides as such, which can also be targets of antibodies and other binding molecules.

### REFERENCE TO RELATED APPLICATIONS

This patent matter is related to and claims priority to provisional patent application U.S. Ser. No. 63/080,009, filed Sep. 17, 2020.

### FIELD OF THE INVENTION

This invention relates generally to cancer antigens.

### BACKGROUND OF THE INVENTION

Human endogenous retroviruses (HERVs) are well-known as genomic repeat sequences, with many copies in the genome, such that approximately 8% of the human genome is of retroviral origin. See, scientific reference 1 below. HERVs originated from thousands of ancient integration events which incorporated retrovirus DNA into germline cells 2. Typically, retroviruses lose infectivity because of the accumulation of mutations. Hence, these genes are predominantly silent and not expressed in normal adult human tissues, except during pathologic conditions such as cancer. The most biologically active HERVs are members of the HERV-K family. HERV-K has a complete sequence capable of expressing all the elements needed for a replication-competent retrovirus (scientific references 3, 4), but remained silent in normal cells. However, in some circumstances, such as in tumors, the inventors and others have reported that expression of HERV-K is activated, and its envelope (Env) protein can be detected in several different types of tumors at much higher levels than in normal tissues. See, scientific references 5-23. This indicates that HERV-K could be an excellent tumor associated antigen and an ideal target for cancer immunotherapy, because it is expressed in tumors and is absent in normal tissues, which minimizes off-target effects.

A significant consideration in developing a cancer therapeutic is the expression profile of the tumor associated antigen, HERV-K is transcriptionally active in germ cell tumors (scientific reference 24), melanoma (scientific reference 25), breast cancer cell lines (T47D) (scientific references 26-28), breast cancer tissues (scientific references 15, 29), and ovarian cancer (scientific reference 13). The inventors specifically identified HERV proteins and sequences in cancer cell lines and patient tumors. The inventors observed the expression of HERVs, especially HERV-K sequences, in breast, Lung, prostate, ovarian, colon, pancreatic, and other solid tumors. See, scientific reference 11, 12, 16, 17, 20, 30-34, They also found that the expression of HERV-K env transcripts in breast cancer was specifically associated with basal breast cancer, a particularly aggressive subtype 20.

Several diagnostic products can be used as companion diagnostics for patient selection. One strategy targets endogenous viral antigens that are found only on cancer cells—not on normal tissues. Viral RNAs are released from these tumors, and both HERV-K RNAs (env or gag) and anti-HERV-K antibodies were discovered by the inventors' group to appear in the circulation of cancer patients. See, scientific references 31-33, 35. These proteins of non-human origin can be exploited as ideal targets for cancer therapy, and as companion diagnostics for therapeutic antibodies that target HERV-K.

The dramatically increased numbers of clinical trials of immunotherapy in multiple cancer types prompted the pursuit of high-efficacy immunotherapy for breast cancer. An improved understanding of the tumor microenvironment of breast cancer is critical to the design of rational and efficient therapy. One problem that has limited the success of therapy against solid tumors is the absence of tumor antigens that are highly expressed in tumor cells but not normal cells.

In the inventors' previous work, the inventors showed that the HERV-K Env protein is commonly expressed on the surface of breast cancer cells 30. Epithelial-mesenchymal transition (EMT) lowers infiltration of CD4 or CD8 T cells in some tumors 36, and HERV-K expression was demonstrated to induce EMT, leading to an increase in cell motility 37, both of which favor tumor dissemination. Scientific publications 10, 33, 37 provide strong evidence that overexpression of HERV-K leads to cancer onset and contributes to cancer progression. A chimeric antigen receptor (CAR) specific for HERV-K env protein (K-CAR) was generated from an anti-HERV-K monoclonal antibody (mAb) (termed 6H5), and anti-metastatic tumor effects of K-CAR therapy were demonstrated in breast cancer and melanoma. Scientific references 33, 35. Importantly, downregulated expression of HERV-K and Ras was revealed in cancer cells treated with either K-CAR T cells or shRNAenv. See, scientific references 10, 33, 38.

### SUMMARY OF THE INVENTION

The inventors found that checkpoint molecule levels in serum and tumor-infiltrating lymphocytes (TILS) are highly correlated to HERV-K antibody titers, especially in aggressive breast cancer patients (patients with invasive ductal carcinoma (IDC) or invasive mammary carcinoma (IMC)). The phenotypic and functional characteristics of TILs in breast cancer are related to HERV-K status, and the combination of checkpoint inhibition and HERV-K antibody therapy could result in better killing efficacy.

The invention provides therapeutic humanized anti-HERV-K antibodies or a fusion thereof consisting of a bispecific T cell engager (BiTE) FOR CD3 and CD8, a DNA-encoded BiTE (DBiTE), or an antibody-drug conjugate (ADC).

In a first embodiment, the invention provides cancer cells overexpressing HERV-K. These cancer cells can be particularly good targets and good models for the anti-HERV-K humanized antibodies and ADCs of the invention, since more antibodies may be bound per cell.

In a second embodiment, the invention provides two humanized antibody clones (HUM1 and HUM2) generated from bacteria and a humanized antibody generated from mammalian cells (hu6H5). Both clones can bind antigens produced from recombinant HERV-K Env surface fusion protein (KSU) and lysates from MDA-MB-231 breast cancer cells. The hu6H5 generated from mammalian cells was compared with our other forms of anti-HERV-K antibodies. The hu6H5 has binding affinity to HERV-K antigen that is similar to murine antibodies (m6H15), chimeric antibodies (cAb), or humanized antibody (HUM1). The hu6H5 antibody induces cancer cells to undergo apoptosis, inhibits cancer cell proliferation, and kills cancer cells that express HERV-K antigen. Importantly, the hu6H5 antibody was demonstrated to reduce tumor viability in mouse MDA-MB-231 xenografts, and notably was able to reduce cancer cell metastasis to lung and lymph nodes. Mice bearing human breast cancer tumors that were treated with these humanized antibodies had prolonged survival compared to control mice that did not receive antibody treatment.

In a third embodiment, the invention provides HERV-K env gene generated from a breast cancer patient as an oncogene which can induce cancer cell proliferation, tumor growth, and metastasis to lungs and lymph nodes. Cells expressing HERV-K showed reduced expression of genes associated with tumor suppression, including Caspases 3 and 9, pRB, SIRT-1 and CIDEA, and increased expression of genes associated tumor formation, including Ras, p-ERK, P-P-38, and beta Catenin.

In a fourth embodiment, the invention provides BiTEs directed against T cell CD3 or CD8 and the tumor-associated antigen HERV-K. The inventors produced such a BiTE, which was comprised of antibodies targeting either CD3 or CD8 and HERV-K (VL-VH 6H5scFv---VH-VLhuCD3 or CD8+c-myc+FLAG) or (VL-VH hu6H5scFv---VH-VLhuCD3 or huCD8+c-myc+FLAG). FLAG-tag, a peptide recognized by an antibody (DYKDDDDK) (SEQ ID NO: 39) and Myc-tag, a short peptide recognized by an antibody (EQKLISEEDL) (SEQ ID NO: 40).

In a fifth embodiment, the invention provides T cells expressing a lentiviral CAR expression vector that bears a humanized or fully human HERV-K scFd.

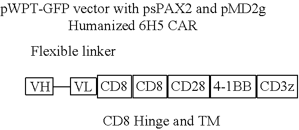

These T-cells effectively lyse and kill tumor cells from several different cancers. Humanized K-CARs expressed from lentiviral vectors are pan-cancer CAR-Ts.

In a sixth embodiment, the invention provides humanized single chain variable fragment (scFv) antibody. This antibody can hind antigens produced from recombinant HERV-K Env surface fusion protein (KSU) and lysates from MDA-MB-231 breast cancer cells. A CAR produced from this humanized say can be cloned into a lentiviral vector. This recombinant vector can be used in combination with therapies, including but are not limited to K-CAR T cells plus checkpoint inhibitors, proinflammatory cytokines such as interleukin (IL)-12 and IL-18, oncolytic viruses, and kinase inhibitors. The kinase inhibitors include but not limited to p-RSK and p-ERK.

In a seventh embodiment, the invention provides HERV-K staining that overlaps in many cases with staining of the serum tumor marker CK. HERV-K can be a CTC marker as well as a target for HERV-K antibody therapy.

In an eighth embodiment, the invention provides HERV-K as a stem cell marker. Targeting of HERV-K can block tumor progression by slowing or preventing growth of cancer stem cells. Targeting of HERV-K with circulating therapeutic antibodies or other therapies can also kill CTCs and prevent metastasis of these circulating cells to distant sites.

In a ninth embodiment, the invention provides that forced overexpression of HERV-K with agents that induce expression of HERV-K by innate immune response (such as Poly I:C treatment) or LTR hypomethylation (such as by 5-Aza) provokes cancer cells to increase production of a target that would make them more susceptible to targeted therapy to include targeted immunotherapy.

In a tenth embodiment, the invention improves an in vivo enrichment technique (IVE: ≈20-fold enhancement) in SCID/beige mice, allowing for rapid expansion and B cell activation. This improved technique can produce many antigen-specific plasmablasts. For donors who have cancer with a higher titer of antibodies, the improved technique uses a protocol with humanized mice (HM) or human tumor mice (HTM) instead SCID/beige mice. For normal donors who do not have cancer and who have no memory B cells, the improved technique uses a protocol with modifications: Mice are treated with cytokine cocktails (days 1, 7, and 14) and boosted by antigens on days 14 and 21, Sera are collected from mice and binding affinity is tested by ELISA. After increased antibody titers are detected, spleens are harvested, analyzed, and used to make hybridomas. Higher antibody titers were detected in mice using an IVE protocol.

In an eleventh embodiment, the invention provides a method to determine cells that not only produce antibodies but are also able to bind antigen and kill cancer cells. This method can efficiently stimulate and expand CD40-B cells to large numbers in high purity (>90%) and induce secretion of their antibodies.

In a twelfth embodiment, the invention provides a method of post-incubation of treated B cells. Glass cover slips are washed and tagged with fluorescent anti-human IgG antibody and read using a microengraving technology to reveal discrete spots that correspond to secretion of antigen-specific antibodies by single B cells.

In a thirteenth embodiment, the invention provides for the development of a platform to determine the binding kinetics and cell-to-cell interactions of every cell in a microwell slab.

In a fourteenth embodiment, the invention strikingly provides significantly enhanced expression of six circulating immune checkpoint proteins in the plasma of breast cancer patients. The invention also provides a marked drop in immune checkpoint protein levels in patients at 6 months or 18 months post-surgery vs. pre-surgery. Importantly, a positive association between soluble immune checkpoint protein molecule levels and HERV-K antibody titers induced by HERV-K expression in the tumor results. HERV-K antibody titers can influence immune checkpoint protein levels in breast cancer. Thus, the expression of HERV-K can control the immune responses of breast cancer patients.

In another aspect, these findings collectively show that the immunosuppressive domain (ISD) of HERV-K is a yet unrecognized immune checkpoint on cancer cells, analogous to the PD-L1 immune checkpoint. In a fifteenth embodiment, the invention provides that blockade of the ISD with immune checkpoint inhibitors of HERV-K, including but not limited to monoclonal antibodies and drugs targeting the ISD of HERV-K, is a cancer immunomodulator therapy that will allow T cells to continue working and unleash immune responses against cancer as well as enhance existing responses, to promote elimination of cancer cells.

In a sixteenth embodiment, the invention provides humanized and fully human (hTab) antibodies targeting HERV-K. These antibodies enhance checkpoint blockade antibody treatment efficacy. Effective combined cancer therapies include but are not limited to combinations of (a) HERV-K humanized or hTAb (1.5 mg/kg), (b) K-CAR, (c) K-BiTE, (d) HERV-K shRNAs or CRISPR/Cas9 genome editing technology to knock down HERV-K gene expression, (e) or preventative or therapeutic HERV-K vaccines, including full-length and truncated HERV-K Env proteins and HERV-K Env peptides. Effective combined cancer therapies include full-length and truncated HERV-K Env proteins and HERV-K Env peptides, combined with factors including but not limited to (a) anti-ICP antibody, (b) cancer chemotherapy, (c) 5-Azacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion, (f) induction of S or G2 phase cell cycle arrest, (g) inhibitors of PI3K/AKT/mTOR or MAPK/ERK signaling pathways, or (f) signal transduction to HIF1α.

In a seventeenth embodiment, the invention provides humanized antibodies targeting HERV-K that can be used for ADCs to deliver the drugs into cancer cells and tumors.

In an eighteenth embodiment, the invention provides antibodies targeting HERV-K that can be used for tumor imaging.

In a nineteenth embodiment, the invention provides a new CAR using hu6H5 scFv.

In a twenty embodiment, the invention provides a new BITE using hu6H5 scFv including CD3 and CD8 BiTEs.

### BRIEF DESCRIPTION OF THE DRAWINGS

FIG. 1 is a Western blot employed to detect the VH chain and VL chain of the humanized anti-HERV-K antibody in an SDS-PAGE gel under reducing conditions. A 49 KDa molecular mass for the VH chain and a 23 KDa molecular mass for the VL chain was detected.

FIG. 2. Size-exclusion chromatography (SEC) separation by size and/or molecular weight was further employed to determine protein expression. Only two peaks were detected and the concentration of peak 2 was greater than 99% of the total combined size of peaks 1 and 2.

FIG. 3. ELISA was used for comparing binding to the HERV-K env target of chimeric 6H5, HUM1 (generated from bacteria), and new hu6H5 (new humanized anti-HERV-K antibody generated from mammalian cells). 1000, 1:1000 dilution; 2000, 1:2000 dilution; 4000, 1:4000 dilution; 8000, 1:8000 dilution.

FIG. 4. Apoptosis assays were employed to determine the cytotoxicity of mouse and humanized anti-HERV-K antibodies toward cancer cells. Cancer cells including MDA-MB-231-pLVXK (231K) (a breast cancer cell line transduced with pLVX vector that expresses HERV-K env protein) or MDA-MB-231-pLVXC (231C) (the same breast cancer cell line transduced with pLVX empty vector) were treated with either m6H5 or hu6H5 (1 or 10 μg per ml) for 4 hours or 16 hours. Annexin V and 7AAD were used to determine the percentage of apoptotic cells. Results after antibody treatment for 4 hours is shown here.

FIG. 5. Live/dead cell viability assays were employed to assess induction of cell death after anti-HERV-K antibody treatment. MDA-MB-231 cells were seeded overnight in 24 well plates. Cells were treated with various antibodies (10 ug/ml) and incubated for 16 hours at 37 C in a cell culture incubator. Calcein Am (4 ul/10 ml media) and Eth-D1 (20 ul/10 ml media), 200 ul per well, were then added and cells were incubated for 30 min at room temperature. EthD-1 penetrates cells with membrane damage and binds to nucleic acids to produce red fluorescence in dead cells. Live cells (green color; Calcein Am) and putative dead cells (red color; EthD-1) were identified using the co-stained Live/Dead Viability Assay. Human IgG or mouse IgG were used control. No red fluorescent cells were observed after treatment with control human or mouse IgG. However, red fluorescent cells were observed in cells treated with humanized or mouse 6H5 anti-HERV-K antibodies.

FIG. 6. An MTS assay was employed to determine inhibition of proliferation of cells treated with hu6H5. Significantly reduced cell proliferation was observed in cells treated with either of the 6H5 antibodies (human or mouse). Inhibition was more prominent in 231K cells that express higher levels of HERV-K than 231C cells that did not express higher HERV-K levels.

FIG. 7. ADCC was employed to determine the mechanism of antibody-induced cell killing. Greater ADCC lysis of cancer cells were observed in cells treated with hu6H5 than with m6H5, with increasing percentages of PBMCs.

FIG. 8. Mice were inoculated with 231K or 231C cells (2 million by subcutaneous injection). The mice were then treated with hu6H5 antibody (n=3; 4 mg/kg, twice per week). Tumor growth was monitored and measured three times per week, and mouse survival was determined. Treatment of the mice with hu6H5 led to longer survival than treatment of another cohort of mice with the control antibody (n=4). Shorter survival was observed in mice inoculated with 231K cells than in mice inoculated with 231C cells, which indicates that overexpression of HERV-K in breast cancer cells shortens tumor-associated survival.

FIG. 9. Tissues from hu6H5 and no antibody treatment groups were stained with H&E and staining results are shown here in 2×, 4×, and 10×. Reduced tumor viability was demonstrated in mice inoculated with 231C cells treated with hu6H5 (20%; B4), compared to the same cells with no antibody treatment (60%; B14; FIG. 9A).

Also, reduced tumor viability was demonstrated in mice inoculated with 231K cells treated with hu6H5 (45%; B1; FIG. 9B), compared to the same cells with no antibody treatment. (Anti-Ki67 and anti-HERV-K mAb (6H5) were used (FIG. 9C). Reduced tumor viability was demonstrated in mice incubated with 231C treated with hu6H5 (20%; bottom panel) compared with control (60%; top panel).

FIG. 10. Metastases to lung and lymph nodes were observed in mice inoculated with 231K cells. Metastases to lung (FIGS. 10A and 10B) or lymph nodes (FIG. 10C) were observed only in mice inoculated with 231K cells. Reduced tumor viability and increased tumor necrosis was detected in lung of mice inoculated with 231K cells and treated with hu6H5 (FIG. 10B). Visibly enlarged lymph nodes were seen in mice inoculated with 231K, but not in mice inoculated with 231C cells. Reduced tumor viability and increased tumor necrosis were detected in lymph nodes from mice inoculated with 231K cells and treated with hu6H5 (KAB) (FIG. 10C; B18; 40%, bottom panel) vs 231K cells with no added antibody (KCON; top panel) (B26>95%). These results show that HERV-K expression is a causal factor for tumor development, and especially for metastasis to distant organ sites. Importantly, our humanized anti-HERV-K antibody can reduce tumor viability, increase tumor necrosis, and decrease metastasis to the lungs and lymph nodes.

FIG. 11. CD3BiTE mediated secretion of IFN gamma from Normal donor PBMCs in the presence of MDA-MB-231 luc cells. 5×10⁻³ cells/well were seeded in 96-well plate. PBMCs from ND #230341 (positive control) and 4 normal donors were used as effector cells. The ratio of the effector/tumor cells was 10/1. 140 μg/ml of CD3BiTE was used. 72 h after plate set-up, the supernatant was harvested for IFN gamma assay.

FIG. 12. FIG. 12A shows images of living (green color) or dead (red color) of MCF-7 cells were treated with PBMCs plus K3Bi. (0 ng/ml; top panels) or (100 ng/ml; bottom panels) for 72 hours. FIG. 12B Significantly increased killing of cancer cells was demonstrated by an LDH release assay in the supernatant of effector: tumor cells (10:1) treated with K3Bi at 0 ng/ml or 100 ng/ml+PBMC for 72 hours. FIG. 12C IFN-γ secretion was significantly increased in three breast cancer cell lines treated with K3Bi (100 ng/ml) for 72 hours. Untreated cells, PBMC only, or BiTE only were used as controls.

FIG. 13. NOD/SCID/IL-2Rγnull (NSG) mice were inoculated with MDA-MB-231 HERV-K-positive breast cancer cells on day zero and dosed with PBMCs (red arrows) or BiTE (black arrows) on the days indicated. Tumor volumes were calculated throughout the study by measuring tumor volumes using a caliper.

FIG. 14. Percentages of CD4⁺ve PBMCs transduced with CAR-A/CAR-B that got stained with K10 labelled AF488 protein are higher than the percentage of naïve T cells that got stained with K10-labelled AF488 protein.

FIG. 15 shows the microengraving process. In FIG. 15A, enriched B cell were mixed with tumor cells and co-cultured for 2 hours to 16 hours in wells covered with a glass slide coated with HERV-K antigen for an immune assay (top right). B cells able to produce antibody and kill tumor cells were retrievable by a CellCelector (bottom right) for RT-PCR and re-cloned to produce antibodies (bottom left). FIG. 15B. Mammospheres produced from tumor tissues (on days 7 and 14) were used as target cells. Autologous PBMCs were stimulated with cocktails for 4 days to enrich the antibody producing B cells. B cells were then co-cultured with tumor target cells. The cover slides coated with HERV-K Env protein were incubated with the co-cultured cells. Putative dead cells (red color) were identified using the co-stained Live/Dead Viability Assay. EthD-1 penetrates cells with membrane damage and binds to nucleic acids to produce red fluorescence in dead cells. An ELISA assay on the cover slide in the same position of the same well was used to detect B cells producing antibodies that can bind. HERV-K Env protein (Red square). FIG. 15C. B cells (red circle; left) that were HERV-K⁺ (green) and IgG⁺ (red) were picked up by a CellCelector. The cell is shown before (top right) and after (bottom right) cell picking.

FIG. 16. In FIG. 16A), ELISPOT was used to detect IFN-γ secreting spleen cells in mice immunized with HERV-K transmembrane (TM) protein (mouse M1 to M4) or PBS (M5 to M6). See FIG. 16B and FIG. 16C. ELISA was used to detect the anti-HERV-K antibody titers in the mice. Higher titers of antibodies were detected in mice treated with KSU Env protein regardless of CpG (FIG. 16B) or CDN (FIG. 16C) status. FIG. 16D Anti-HERV-K antibody titers were detected by ELISA in HTM models inoculated with MDA-MB-231 (HTM1) or MDA-MB-468 (HTM2) and with HM (1-2) immunized with HERV-K SU Env protein using anti-human IgG mAb.

FIG. 17 Scheme of in vivo HERV-K-driven plasmablast differentiation in human-SCID chimeras. 50 million PBMCs from subjects premixed with HERV-K protein (100 μg) in vitro, are intrasplenically injected into humanized mice on day 0. Cytokine cocktails (BAFF: 50 μg, IL-2: 50 ng, IL-6: 50 ng, and IL-21: 50 ng) are injected intraperitoneally on days 1, 4, and 7. HERV-K Env protein (100 μg) will be boosted intraperitoneally on day 2. IgG⁺, CD38⁺, and HERV-K⁺ will be sorted by flow cytometry or a microengraving platform for subsequent analysis. Half the splenocytes are used to generate hybridomas with an MFP-2 fusion partner. An ELISA assay was used to detect anti-ZIKV Env antibodies from hybridoma clones. Supernatant from each hybridoma clone (100 μl) was added and incubated for one hour. Goat-anti-human IgG/A/M-HRP antibody was then added (1:4,000 dilution), followed by incubation for another hour. High titers of antibodies were demonstrated in some hybridoma clones and from donor 322336. Supernatant obtained from anti-flavivirus 4G2 mAb was used as a positive control (D1-4G2-4-15; ATCC HB-112).

FIG. 18. In FIG. 18A, the percentages of CD33, CD3, and CD 19⁺ cells were quantified in huCD45⁺ cells obtained at four weeks post-inoculation of TNBC PDX cells, and in the MDA-MB-231 HTM model at seven weeks post-inoculation, with co-implantation of CD34⁺ hematopoietic stem cells. FIG. 18B. Flow data from spleen cells at week 7 post-inoculation with MDA-MB-231 cells is shown. FIG. 18C. Immunofluorescence staining was used to detect the expression of HERV-K using anti-HERV-K mAb 6H5 (green) in an MDA-MB-231 tumor obtained from an HIM. F-actin (red) was used as the control (two left panels). huCD3⁺ cells (green color) were also detected in tumor tissues (two right panels). FIG. 18D. Anti-HERV-K antibody titers were detected by ELISA in HTM models inoculated with MDA-MB-231 (HTM1) or MDA-MB-468 (HTM2) and with HM1 and FLM2 immunized with HERV-K SU Env protein using anti-human IgG mAb.

FIG. 19 illustrates the baseline immune status in relation to HERV-K status in breast cancer patients: combined HERV-K and immune checkpoint assays. Expression of soluble immune checkpoint proteins was determined by Luminex assay in breast cancer patients including DCIS and aggressive breast cancer vs. normal donors. FIG. 19A shows the comparison of expression of six ICPs in DCIS, aggressive breast cancer (aBC), and normal female donors. A striking finding was a significantly enhanced expression of the six circulating ICPs in the plasma of breast cancer patients FIG. 19A. FIG. 19B (a-c): A further finding was a marked drop in immune checkpoint protein levels in patients at 6 months (B; Timepoint 2) or 18 months (data not shown) post-surgery vs. pre-surgery (Timepoint 1). Importantly, a positive association between soluble ICP molecule levels and HERV-K antibody titers induced by HERV-K expression in the tumor was observed (FIG. 19C), suggesting that HERV-K antibody titers would influence ICP levels in breast cancer. The expression of HERV-K can thus control immune responses of breast cancer patients.

FIG. 20. In FIG. 20A, an immunoblot assay was used to demonstrate 6H5 conjugation with r-Gel. FIG. 20B. Delivery of the recombinant gelonin toxin (r-Gel) was observed in HERV-K positive cancer cells using the anti-HERV-K 6H5-rGel ADC. Surface and cytoplasmic expression of HERV-K in DOV13 ovarian cancer cells was detected using anti-HERV-K 6H5 mAb. Furthermore, r-Gel expression was detected in DOV13 cells using anti-rGel antibodies post-4 hours treatment. FIG. 20C. HERV-K env protein (6H5; red) or rGel signals (green) were detected in SKBr3, MCF-7, and MDA-MB-231 breast cancer cells after 1 hour internalization. The yellow-orange color indicates a colocalization of HERV-K env protein and rGel toxin in the target cell cytoplasm (right panels). Antitumor effects were compared in mice inoculated with MDA-MB-231 cells treated with 6H5 (p=0.0052), and 6H5-r-Gel (p<0.0001) compared with mice treated with control IgG (FIG. 20D).

FIG. 21 Delivery of gold nanoparticles (GNPs) was demonstrated in various HER positive breast cancer cell lines in vitro and in vivo. GNPs (dark spots) were detected in MDAMB231 cells in vitro using TEM after 2 hours incubation with naked GNP (FIG. 21A) or 6H5-GNP (FIG. 21B). GNPs were detected in MDAMB231 tumor (FIG. 21C) or SKBr3 tumor (FIG. 21D) 24 hours post-intravenous injection with 6H5-GNP or 6H5scFv-GNP in the tail vein of mice, using a silver enhancement assay. (E/F) GNPs (white arrows) were detected by TEM in MDAMB231 cells of tumors isolated from mice 24 hours post-intravenous-injection with 6H5-GNP. HERV viral particles (green arrows) were observed adjacent to tumor cells.

FIG. 22A higher density of 6H5 mAb was detected in tumor nodules from mice 24 hours post-intravenous-injection with 6H5-Alexa647 (red color) by in vivo imaging using a Nuance system.

FIG. 23 Mice were immunized with 5 MAPS identified from BC patient serum samples and affinities of antibodies produced in the mice toward various HERV viral proteins including HERV-K SU envelope protein (K10G15), ERV3 (E3G4), Rec, Np9, and HERV-K TM envelope protein were determined. Anti-HERV-KSU antibodies were demonstrated in sera of three mice, and an antibody sequence was generated from hybridoma cells generated from mouse #2.

### DETAILED DESCRIPTION OF THE INVENTION

### Utility

This specification provides methods for generated a humanized anti-HERV-K antibody. Anti-tumor effects of hu6H5 were demonstrated in vitro and in vivo.

This invention provides methods for treating patients suffering from cancer. In a twentieth embodiment, the invention provides to a method of treating cancer comprising administering a therapeutic humanized anti-HERV-K antibody or a fusion thereof consisting of a CAR, a BiTE or an ADC, a cancer vaccine, and optionally combine with one or more immune checkpoint blockers. Each of these therapeutics individually target the immune system. In a twenty-first embodiment, the methods of the invention inhibit metastases. In a twenty-second embodiment, the methods of the invention reduce tumor size. In a twenty-third embodiment, the methods of the invention inhibit the growth of tumor cells. In a twenty-fourth embodiment, the methods of the invention detect cancer and cancer metastasis.

### Definitions

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skill in the molecular biology art. For any apparent discrepancy between the meaning of a term in the art and a definition provided in this specification, the meaning provided in this specification shall prevail.
“5-Aza” has the biotechnological art-recognized meaning of 5-azacytidine.
“6H5” has the biotechnological art-recognized meaning of a murine anti-HERV-K monoclonal antibody developed in our laboratory.
“About” will be understood by persons of ordinary skill in the molecular biological art and will vary to some extent depending on the context in which it is used. If there are uses of the term which are not clear to persons of ordinary skill in the molecular biological art given the context in which it is used, “about” will mean up to plus or minus 10% of the value.
“Antibody-drug conjugate (ADC)” has the biotechnological art-recognized meaning of highly potent biological drugs built by attaching a small molecule anticancer drug or another therapeutic agent to an antibody, with either a permanent or a labile linker. The antibody targets a specific antigen only found on target cells.
“B7 family” has the biotechnological art-recognized meaning of inhibitory ligands with undefined receptors. The B7 family encompasses B7-H3 and B7-H4, both upregulated on tumor cells and tumor infiltrating cells. The complete hB7-H3 and hB7-H4 sequence can be found under GenBank Accession Nos. Q5ZPR3 and AAZ17406, respectively.
“BiTE” has the biotechnological art-recognized meaning of a bispecific T cell engager, A BiTE means a recombinant bispecific protein that has two linked scFvs from two different antibodies, one targeting a cell-surface molecule on cells (for example, CD3ε) and the other targeting antigens on the surface of malignant cells. The two scFvs are linked together by a short flexible linker. The term DNA-encoded BiTE (DBiTE) includes any BiTE-encoding DNA plasmid that can be expressed in vivo.
“Cancer antigen” or “tumor antigen” has the biotechnological art-recognized meaning of (i) tumor-specific antigens, (ii) tumor-associated antigens, (iii) cells that express tumor-specific antigens, (iv) cells that express tumor-associated antigens, (v) embryonic antigens on tumors, (vi) autologous tumor cells, (vii) tumor-specific membrane antigens, (viii) tumor-associated membrane antigens, (ix) growth factor receptors, (x) growth factor ligands, and (xi) any other type of antigen or antigen-presenting cell or material that is associated with a cancer.
“Combination therapy” embraces administration of each agent or therapy in a sequential manner in a regimen that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements can be administered at separate times and/or by different routes, but which act in combination to provide a beneficial effect by co-action or pharmacokinetic and pharmacodynamics effect of each agent or tumor treatment approaches of the combination therapy.
“CTL” has the biotechnological art-recognized meaning of cytolytic T cell or cytotoxic T cell.
“Cytotoxic T Lymphocyte Associated Antigen-4 (CTLA-4)” is a T cell surface molecule and is a member of the immunoglobulin superfamily. This protein downregulates the immune system by binding to CD80 and CD86. The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. P16410.
“Derived from” a designated polypeptide or protein has the biotechnological art-recognized meaning of the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the in the molecular biological art as having its origin in the sequence. Polypeptides derived from another peptide can have one or more mutations relative to the starting polypeptide, e.g., one or more amino acid residues which were substituted with another amino acid residue, or which has one or more amino acid residue insertions or deletions. A polypeptide can comprise an amino acid sequence which is not naturally occurring. Such variants necessarily have less than 100% sequence identity or similarity with the starting molecule. In some embodiments, the peptides are encoded by a nucleotide sequence. Nucleotide sequences of the invention can be useful for several applications, including cloning, gene therapy, protein expression and purification, mutation introduction, DNA vaccination of a host in need thereof, antibody generation for, e.g., passive immunization, PCR, primer and probe generation, and the like.
“Effector cell” has the biotechnological art-recognized meaning of an immune cell which is involved in the effector phase of an immune response, as opposed to the cognitive and activation phases of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express specific Fe receptors (FcRs) and carry out specific immune functions.
“Epitope” means a protein determinant capable of specific binding to an antibody. Epitopes usually consist of surface groupings of molecules such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents. The epitope can comprise amino acid residues directly involved in the binding (also called immunodominant component of the epitope) and other amino acid residues, which are not directly involved in the binding, such as amino acid residues which are effectively blocked by the specifically antigen binding peptide (in other words, the amino acid residue is within the footprint of the specifically antigen binding peptide).
“FACS” refers fluorescence activated cell sorting.
“HERV” has the biotechnological art-recognized meaning of human endogenous retrovirus, “HERV-K” has the biotechnological art-recognized meaning of the HERV-K family of endogenous retroviruses. The “human endogenous retrovirus” (HERV) is a retrovirus that is present in the form of proviral DNA integrated into the genome of all normal cells and is transmitted by Mendelian inheritance patterns. “HERV-X,” where “X” is an English letter, has the biotechnological art-recognized meaning of other families of HERVs. “Env” has the biotechnological art-recognized meaning of the viral envelope protein. “KSU” has the biotechnological art-recognized meaning of HERV-K envelope surface fusion protein, and “KTM” has the biotechnological art-recognized meaning of HERV-K Env transmembrane protein. “env” has the biotechnological art-recognized meaning of the viral envelope RNA. pLVXK has the biotechnological art-recognized meaning of an HERV-K expression vector. The terms MDA-MB-231 pLVXK or 231-K refer to MDA-MB-231 cells that were transduced with pLVXK. pLVXC has the biotechnological art-recognized meaning of a control expression vector only. The terms MDA-MB-231 pLVXC or 231-C refer to MDA-MB-231 cells that were transduced with pLVXC. HERV-K is expressed on many tumor types, including, but not limited to, melanoma (Muster et ah, 2003; Buscher et al., 2005; Li et al, 2010; Reiche et ah, 2010; Serafino et al, 2009), breast cancer (Patience et al, 1996; Wang-Johanning et al, 2003; Seifarth et al, 1995), ovarian cancer (Wang-Johanning et al., 2007), lymphoma (Contreras-Galindo et al., 2008), and teratocarcinama (Bieda et al,, 2001; Lower et al., 1993). Furthermore, infected cells, including those infected by HIV (Jones et al., 2012), also express HERV-K. This provides an attractive opportunity that one CAR design targeting HERV-K may be used to treat a variety of cancers and infections.
“HM” has the biotechnological art-recognized meaning of humanized mice and “HTM” has the biotechnological art-recognized meaning of human tumor mice.
“hTAb” has the biotechnological art-recognized meaning of a fully human tumor antibody.
“Human endogenous retrovirus-K,” “HERV-K,” “HERV,” “human endogenous retrovirus,” “endogenous retrovirus,” and “ERV” include any variants, isoforms and species homologs of endogenous retroviruses which are naturally expressed by cells or are expressed on cells transfected with endogenous retroviral genes.
“ICP” has the biotechnological art-recognized meaning of immune checkpoint.
“IHC” has the biotechnological art-recognized meaning of immunohistochemistry,
“ILC” has the biotechnological art-recognized meaning of invasive lobular carcinoma. “DCIS” has the biotechnological art-recognized meaning of ductal carcinoma in situ. “IDC” has the biotechnological art-recognized meaning of invasive ductal carcinoma.
“Immune cell” is a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).
“Immune checkpoint blocker” has the biotechnological art-recognized meaning of a molecule that totally or partially reduces, inhibits, interferes with, or modulates one or more checkpoint proteins. In sonic embodiments, the immune checkpoint blocker prevents inhibitory signals associated with the immune checkpoint. In some embodiments, the immune checkpoint Mocker is an antibody, or fragment thereof that disrupts inhibitory signaling associated with the immune checkpoint. In some embodiments, the immune checkpoint blocker is a small molecule that disrupts inhibitory signaling. In some embodiments, the immune checkpoint blocker is an antibody, fragment thereof, or antibody mimic, that prevents the interaction between checkpoint blocker proteins, e.g., an antibody, or fragment thereof, that prevents the interaction between PD-1 and PD-L1. In some embodiments, the immune checkpoint blocker is an antibody, or fragment thereof, that prevents the interaction between CTLA-4 and CD80 or CD86. In some embodiments, the immune checkpoint blocker is an antibody, or fragment thereof, that prevents the interaction between LAG3 and its ligands, or TIM-3 and its ligands. The checkpoint blocker can also be in the form of the soluble form of the molecules (or variants thereof) themselves, e.g., a soluble PD-L1 or PD-L1 fusion.
“Immune checkpoint” has the biotechnological art-recognized meaning of co-stimulatory and inhibitory signals that regulate the amplitude and quality of T cell receptor recognition of an antigen. In some embodiments, the immune checkpoint is an inhibitory signal. In some embodiments, the inhibitory signal is the interaction between PD-1 and PD-L1. In some embodiments, the inhibitory signal is the interaction between CTLA-4 and CD80 or CD86 to displace CD28 binding. In some embodiments the inhibitory signal is the interaction between LAG3 and MHC class II molecules. In some embodiments, the inhibitory signal is the interaction between TIM3 and galectin 9.
“In vivo” has the biotechnological art-recognized meaning of processes that occur in a living organism. The term “mammal” or “subject” or “patient” as used herein includes both humans and non-humans and includes, but is not limited to, humans, non-human primates, canines, felines, rodents, bovines, equines, and pigs.
“Inhibits growth” (e.g. referring to cells, such as tumor cells) is intended to include any measurable decrease in the cell growth when contacted with HERV-K specific therapeutic agents as compared to the growth of the same cells not in contact with the HERV-K specific therapeutic agents, e.g., the inhibition of growth of a cell culture by at least about 10%, 20%, 30%, 40%, 50%, 60%, 70%, 80%, 90%, 99%, or 100%. Such a decrease in cell growth can occur by a variety of mechanisms exerted by the anti-HERV-K agents, either individually or in combination, e.g., apoptosis.
“ISD” has the biotechnological art-recognized meaning of immunosuppressive domain.
“K-CAR” or “HERV-Kenv CAR” has the biotechnological art-recognized meaning of a HERV-K envelope gene (surface or transmembrane) chimeric antigen receptor (CAR) genetic construct. The term “HERV-Kenv CAR-T cells” or “K-CAR-T cells” has the biotechnological art-recognized meaning of T cells that were transduced with a K-CAR or HERV-Kenv CAR lentiviral or Sleeping Beauty expression system.
“KD” has the biotechnological art-recognized meaning of knockdown, usually by an shRNA.
“Linked,” “fused,” or “fusion,” are used interchangeably. These terms refer to the joining together of two more elements or components or domains, by whatever means including chemical conjugation or recombinant means. Methods of chemical conjugation (e.g., using heterobifunctional crosslinking agents) are known in the art.
“Linker” or “linker domain” has the biotechnological art-recognized meaning of a sequence which connects two or more domains (e.g., a humanized antibody targeting HERV-K and an antibody targeting a T cell protein) in a linear sequence. The constructs suitable for use in the methods disclosed herein can use one or more “linker domains,” such as polypeptide linkers. “Polypeptide linker” has the biotechnological art-recognized meaning of a peptide or polypeptide sequence (e.g., a synthetic peptide or polypeptide sequence) which connects two or more domains in a linear amino acid sequence of a polypeptide chain. Such polypeptide linkers can provide flexibility to the polypeptide molecule. The polypeptide linker can be used to connect (e.g., genetically fuse) one or more Fc domains and/or a drug.
“Lymphocyte Activation Gene-3 (LAG3)” is an inhibitory receptor associated with inhibition of lymphocyte activity by binding to MHC class II molecules. This receptor enhances the function of Treg cells and inhibits CD8+ effector T cell function. “LAG3” as used herein includes human LAG3 (hLAG3), variants, isoforms, and species homologs of hLAG3, and analogs having at least one common epitope. The complete hLAG3 sequence can be found under GenBank Accession No. P18627.
“Mammosphere” has the biotechnological art-recognized meaning of breast or mammary cells cultured under non-adherent non-differentiating conditions that form discrete clusters of cells.
“Nucleic acid” has the biotechnological art-recognized meaning of deoxyribonucleotides or ribonucleotides and polymers thereof in either single-stranded or double-stranded form. Unless specifically limited, encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues. See Batzer et al., Nucleic Acid Res., 19, 5081 (1991); Ohtsuka et al., Biol. Chem., 260, 2605-2608 (1985); and Rossolini et al., Mot. Cell. Probes, 8, 91-98 (1994). For arginine and leucine, modifications at the second base can also be conservative. The term nucleic acid is used interchangeably with gene, cDNA, and mRNA encoded by a gene.
“PBMC” has the biotechnological art-recognized meaning of peripheral blood mononuclear cell.
“PDX” has the biotechnological art-recognized meaning of a patient derived xenograh. A PDX is typically produced by transplanting human tumor cells or tumor tissues into an immunodeficient murine model of human cancer.
“Percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequences relative to the reference sequence, based on the designated program parameters. Optimal alignment of sequences for comparison can be conducted, e.g., by the local homology algorithm of Smith & Waterman, Adv. Appl. Math., 2, 482 (1981), by the homology alignment algorithm of Needleman & Wunsch, J. Mol. Biol., 48, 443 (1970), by the search for similarity method of Pearson & Lipman, Proc. Natl. Acad. Sci,, U.S.A., 85, 2444 (1988), by computerized implementations of these algorithms (GAP, BESHERV-KIT, FASTA, and HERV-KASTA in the Wisconsin Genetics Software Package, Genetics Computer Group, 575 Science Dr., Madison, WI, USA), or by visual inspection. One example of an algorithm that is suitable for determining percent sequence identity and sequence similarity is the BLAST algorithm, which is described in Altschul et aL, J. Mol. Biol. 215, 403-410 (1990). Software for performing BLAST analyses is publicly available through the National Center for Biotechnology Information website.
“Pharmaceutically acceptable” generally means those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.
“Programmed Death Ligand-1 (PD-L1)” is one of two cell surface glycoprotein ligands for PD-1 (the other being PD-L2) that downregulates T cell activation and cytokine secretion upon binding to PD-1. “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.
“Programmed Death-1 (PD-1)” receptor has the biotechnological art-recognized meaning of an immuno-inhibitory receptor belonging to the CD28 family. PD-1 is expressed predominantly on previously activated T cells in vivo, and binds to two ligands, PD-L1 and PD-L2. “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773.
“Recombinant host cell” (or simply “host cell”) has the biotechnological art-recognized meaning of a cell into which an expression vector was introduced. Such terms refer not only to the particular subject cell, but also to the progeny of such a cell. Because some modifications can occur in succeeding generations due to either mutation or environmental influences, such progeny might not, in fact, be identical to the parent cell, but are still included within the scope of “host cell” as used herein. Recombinant host cells include, for example, transfectomas, such as CHO cells, HEK293 cells, NS/0 cells, and lymphocytic cells.
“scFv” has the biotechnological art-recognized meaning of single chain variable fragment.
“SU” has the biotechnological art-recognized meaning of the HERV-K surface protein.
“Sufficient amount” or “amount sufficient to” means an amount sufficient to produce a desired effect, e.g., an amount sufficient to reduce the size of a tumor.
“Synergy” or “synergistic effect” regarding an effect produced by two or more individual components has the biotechnological art-recognized meaning of a phenomenon in which the total effect produced by these components, when utilized in combination, is greater than the sum of the individual effects of each component acting alone.
“T Cell Membrane Protein-3 (TIM3)” is an inhibitory receptor involved in the inhibition of lymphocyte activity by inhibition of TH1 cells responses. Its ligand is galectin 9, which is upregulated in various types of cancers. “TIM3” as used herein includes human TIM3 (hTIM3), variants, isoforms, and species homologs of hTIM3, and analogs having at least one common epitope. The complete hTIM3 sequence can be found under GenBank Accession No. Q8TDQo.
“T cell” has the biotechnological art-recognized meaning of a CD4+ T cell or a CD8+ T cell. The term T cell encompasses TH1 cells, TH2 cells and TH17 cells.
“Therapeutically effective amount” is an amount that is effective to ameliorate a symptom of a disease. A therapeutically effective amount can be a “prophylactically effective amount” as prophylaxis can be considered therapy.
“TM” has the biotechnological art-recognized meaning of the HERV-K transmembrane protein.
“TNBC” has the biotechnological art-recognized meaning of triple-negative breast cancer.
“Transgenic non-human animal” has the biotechnological art-recognized meaning of a non-human animal having a genome comprising one or more human heavy and/or light chain transgenes or transchromosomes (either integrated or non-integrated into the animal's natural genomic DNA) and which can express fully human antibodies. For example, a transgenic mouse can have a human light chain transgene and either a human heavy chain transgene or human heavy chain transchromosome, such that the mouse produces human anti-HERV-K antibodies when immunized with HERV-K antigen and/or cells expressing HERV-K. The human heavy chain transgene can be integrated into the chromosomal DNA of the mouse, as is the case for transgenic mice, for instance HuMAb mice or the human heavy chain transgene can be maintained extrachromosomally, as is the case for transchromosomal KM mice as described in WO02/43478. Such transgenic and transchromosomal mice (collectively referred to herein as “transgenic mice”) can produce multiple isotypes of human mAbs to a given antigen (such as IgG, IgA, IgM, IgD, or IgE) by undergoing V-D-J recombination and isotype switching. Transgenic, nonhuman animal can also be used for production of antibodies against a specific antigen by introducing genes encoding such specific antibody, for example by operatively linking the genes to a gene which is expressed in the milk of the animal.
“Treatment” means the administration of an effective amount of a therapeutically active compound of the invention with the purpose of easing, ameliorating, arresting, or eradicating (curing) symptoms or disease states.
“Vector” has the biotechnological art-recognized meaning of a nucleic acid molecule capable of transporting another nucleic acid to which it was linked. One type of vector is a “plasmid,” which has the biotechnological art-recognized meaning of a circular double-stranded DNA loop into which additional DNA segments can be ligated. Another type of vector is a viral vector, wherein additional DNA segments can be ligated into the viral genome. Some vectors are capable of autonomous replication in a host cell into which they are introduced (for instance bacterial vectors having a bacterial origin of replication and episomal mammalian vectors). Other vectors (such as non-episomal mammalian vectors) can be integrated into the genome of a host cell upon introduction into the host cell, and thereby are replicated along with the host genome. Moreover, some vectors can direct the expression of genes to which they are operatively linked. Such vectors are referred to herein as “recombinant expression vectors” (or simply, “expression vectors”). Expression vectors useful in recombinant DNA techniques are often in the form of plasmids. In this specification, the terms “plasmid” and “vector” are used interchangeably as the plasmid is the most commonly used form of vector. However, the invention is intended to include such other forms of expression vectors, such as viral vectors (such as replication-defective retroviruses, adenoviruses, and adeno-associated viruses), which serve equivalent functions.

Over the last 15 years, the development of cancer therapeutic antibodies, such as Herceptin® (trastuzumab), Avastin® (bevacizumab), Erbitux® (cetuximab), and others saved many tens of thousands of lives worldwide. In particular, the treatment of HER2-positive metastatic breast or ovarian cancer using trastuzumab has dramatically changed patient outcomes 40. Antibody therapeutics offer distinct advantages relative to small molecule drugs, namely: (i) defined mechanisms of action; (ii) higher specificity and fewer-off target effects; and (iii) predictable safety and toxicological profiles 41, 42. Currently >200 antibody therapeutics are in clinical trials in the United States. As extensive studies with anti-Her2 and anti-EGFR monoclonals attest, only a few antibodies out of many thousands identified based on their ability to bind to their molecular target with high affinity exhibit properties required for clinically effective cancer cell killing 41. The efficacy of therapeutic antibodies results primarily from their ability to elicit potent tumor cytotoxicity either via direct induction of apoptosis in target cells or through effector-mediated functions like antibody dependent cell-mediated cytotoxicity (ADCC) and complement dependent cytotoxicity (CDC) 41, 42.

The major methodologies for antibody isolation are: (i) in vitro screening of libraries from immunized animals or from synthetic libraries using phage or microbial display 43-45, and (ii) isolation of antibodies following B cell immortalization or cloning 46-48. These methodologies suffer from either or both of the following drawbacks, severely limiting the numbers of unique antibodies that can be isolated: (i) the need for extensive screening to isolate even small numbers of high-affinity antibodies and (ii) immune responses against these antibodies when injected into humans. Thus, regardless of the methodology used for screening/isolation of therapeutic monoclonal antibodies (mAbs), the translation rate from discovery to clinic is inefficient and laborious 47, 49.

One advance that accelerated the approval of therapeutic mAbs was the generation of humanized antibodies by the complementary-determining region (CDR) grafting technique. See scientific reference 10. In CDR grafting, non-human antibody CDR sequences are transplanted into a human framework sequence to maintain target specificity.

### Humanized Antibody and Antibody-Drug Conjugate (ADC) Pharmaceutical Compositions

Cancer cells overexpressing HERV-K may be particularly good targets for the anti-HERV-K humanized antibodies and ADCs of the invention, since more antibodies may be bound per cell. Thus, in a twenty-fifth embodiment, a cancer patient to be treated with anti-HERV-K humanized antibodies or ADCs of the invention is a patient, e.g., a breast cancer, ovarian cancer, pancreatic cancer, lung cancer or colorectal cancer patient who was diagnosed to have overexpression of HERV-K in their tumor cells.

Upon purifying anti-HERV-K humanized antibodies or ADCs they may be formulated into pharmaceutical compositions using well known pharmaceutical carriers or excipients.

The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed. (Mack Publishing Co., Easton, Pa., 1995).

The pharmaceutically acceptable carriers or diluents as well as any other known adjuvants and excipients should be suitable for the humanized antibodies or ADCs of the invention and the chosen mode of administration. Suitability for carriers and other components of pharmaceutical compositions is determined based on the lack of significant negative impact on the desired biological properties of the chosen compound or pharmaceutical composition of the invention (e.g., less than a substantial impact (10% or less relative inhibition, 5% or less relative inhibition, etc.)) on antigen binding.

A pharmaceutical composition of the invention may also include diluents, fillers, salts, buffers, detergents (e.g., a nonionic detergent, such as Tween-20 or Tween-80), stabilizers (e.g., sugars or protein-free amino acids), preservatives, tissue fixatives, solubilizers, and/or other materials suitable for inclusion in a pharmaceutical composition.

The actual dosage levels of the humanized antibodies or ADCs in the pharmaceutical compositions of the invention may be varied to obtain an amount of the humanized antibodies or ADCs which is effective to achieve the desired therapeutic response for a particular patient, composition, and mode of administration, without being toxic to the patient. The selected dosage level will depend upon a variety of pharmacokinetic factors including the activity of the particular compositions of the invention used, the route of administration, the time of administration, the rate of excretion of the particular compound being used, the duration of the treatment, other drugs, compounds and/or materials used in combination with the particular compositions used, the age, sex, weight, condition, general health and prior medical history of the patient being treated, and like factors welt known in the medical arts.

The pharmaceutical composition may be administered by any suitable route and mode. Suitable routes of administering the humanized antibodies or ADCs of the invention are well known in the art and may be selected by those of ordinary skill in the molecular biological art.

In a twenty-sixth embodiment, the pharmaceutical composition of the invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In a twenty-seventh embodiment, the pharmaceutical composition is administered by intravenous or subcutaneous injection or infusion.

Pharmaceutically acceptable carriers include any and all suitable solvents, dispersion media, coatings, antibacterial and antifungal agents, isotonicity agents, antioxidants and absorption delaying agents, and the like that are physiologically compatible with humanized antibodies or ADCs of the invention.

Examples of suitable aqueous and nonaqueous carriers which may be used in the pharmaceutical compositions of the invention include water, saline, phosphate buffered saline, ethanol, dextrose, polyols (such as glycerol, propylene glycol, polyethylene glycol, and the like), and suitable mixtures thereof, vegetable oils, such as olive oil, corn oil, peanut oil, cottonseed oil, and sesame oil, carboxymethyl cellulose colloidal solutions, tragacanth gum and injectable organic esters, such as ethyl oleate, and/or various buffers. Other carriers are well known in the pharmaceutical arts.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except as far as any conventional media or agent is incompatible with the anti-HERV-K humanized antibodies or ADCs of the invention, use thereof in the pharmaceutical compositions of the invention is contemplated.

Proper fluidity may be maintained, for example, using coating materials, such as lecithin, by the maintenance of the required particle size for dispersions, and using surfactants.

The pharmaceutical compositions of the invention may also comprise phamiaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the invention may also comprise isotonicity agents, such as sugars, polyalcohols, such as mannitol, sorbitol, glycerol, or sodium chloride in the compositions.

The pharmaceutical compositions of the invention may also contain one or more adjuvants appropriate for the chosen route of administration such as preservatives, wetting agents, emulsifying agents, dispersing agents, preservatives, or buffers, which may enhance the shelf life or effectiveness of the pharmaceutical composition. The anti-HERV-K humanized antibodies or ADCs of the invention may be prepared with carriers that will protect the compound against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the molecular biological art. Methods for the preparation of such formulations are generally known to those skilled in the molecular biological art. See e.g., Sustained and Controlled Release Drug Delivery Systems, J. R. Robinson, ed. (Marcel Dekker, Inc., New York, 1978).

In a twenty-eighth embodiment, the anti-HERV-K humanized antibodies or ADCs of the invention may be formulated to ensure proper distribution in vivo. Pharmaceutically acceptable carriers for parenteral administration include sterile aqueous solutions or dispersions and sterile powders for the extemporaneous preparation of sterile injectable solutions or dispersion. The use of such media and agents for pharmaceutically active substances is known in the art. Except as far as any conventional media or agent is incompatible with the active compound, use thereof in the pharmaceutical compositions of the invention is contemplated. Supplementary active compounds may also be incorporated into the compositions.

Pharmaceutical compositions for injection must typically be sterile and stable under the conditions of manufacture and storage. The composition may be formulated as a solution, micro-emulsion, liposome, or other ordered structure suitable to high drug concentration. The carrier may be an aqueous or nonaqueous solvent or dispersion medium containing for instance water, ethanol, polyols (such as glycerol, propylene glycol, polyethylene glycol, and the like), and suitable mixtures thereof, vegetable oils, such as olive oil, and injectable organic esters, such as ethyl oleate. The proper fluidity may be maintained, for example, using a coating such as lecithin, by the maintenance of the required particle size for dispersion and using surfactants. In many cases, it will be preferable to include isotonic agents, for example, sugars, polyalcohols such as glycerol, mannitol, sorbitol, or sodium chloride in the composition. Prolonged absorption of the injectable compositions may be brought about by including in the composition an agent that delays absorption, for example, monostearate salts and gelatin. Sterile injectable solutions may be prepared by incorporating the anti-HERV-K humanized antibodies or ADCs in the required amount in an appropriate solvent with one or a combination of ingredients, e.g., as enumerated above, as required, followed by sterilization microfiltration. Generally, dispersions are prepared by incorporating the anti-HERV-K humanized antibodies or ADCs into a sterile vehicle that contains a basic dispersion medium and the required other ingredients e.g., from those enumerated above. For sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

Sterile injectable solutions may be prepared by incorporating the anti-HERV-K humanized antibodies or ADCs in the required amount in an appropriate solvent with one or a combination of ingredients enumerated above, as required, followed by sterilization microfiltration. Generally, dispersions are prepared by incorporating the anti-HERV-K humanized antibodies or ADCs into a sterile vehicle that contains a basic dispersion medium and the required other ingredients from those enumerated above. For sterile powders for the preparation of sterile injectable solutions, examples of methods of preparation are vacuum drying and freeze-drying (lyophilization) that yield a powder of the anti-HERV-K humanized antibodies or ADCs plus any additional desired ingredient from a previously sterile-filtered solution thereof.

The pharmaceutical composition of the invention may contain one anti-HERV-K humanized antibodies or ADCs of the invention or a combination of anti-HERV-K humanized antibodies or ADCs of the invention.

The efficient dosages and the dosage regimens for the anti-HERV-K humanized antibodies or ADCs depend on the disease or condition to be treated and may be determined by the persons skilled in the molecular biological art. An exemplary, non-limiting range for a therapeutically effective amount of a compound of the invention is about 0.1-100 mg/kg, such as about 0.1-50 mg/kg, for example about 0.1-20 mg/kg, such as about 0.1-10 mg/kg, such as about 0.5-5 mg/kg, for instance about 5 mg/kg, such as about 4 mg/kg, or about 3 mg/kg, or about 2 mg/kg, or about 1 mg/kg, or about 0.5 mg/kg, or about 0.3 mg/kg. An exemplary, non-limiting range for a therapeutically effective amount of an anti-HERV-K humanized antibodies or ADCs of the invention is about 0.02-30 mg/kg, such as about 0.1-20 mg/kg, or about 0.5-10 mg/kg, or about 0.5-5 mg/kg, for example about 1-2 mg/kg, in particular of the antibodies 011, 098, 114 or 111 as disclosed herein.

A physician having ordinary skill in the molecular biological art may readily determine and prescribe the effective amount of the pharmaceutical composition required. For example, the physician could start doses of the anti-HERV-K humanized antibodies or ADCs used in the pharmaceutical composition at levels lower than that required to achieve the desired therapeutic effect and gradually increase the dosage until the desired effect is achieved. A suitable daily dose of a composition of the invention will be that amount of the compound which is the lowest dose effective to produce a therapeutic effect. Such an effective dose will generally depend upon the factors described above. Administration can be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for an anti-HERV-K humanized antibodies or ADCs of the invention to be administered alone, it is preferable to administer the anti-HERV-K humanized antibodies or ADCs as a pharmaceutical composition as described above.

In a twenty-ninth embodiment, the anti-HERV-K humanized antibodies or ADCs may be administered by infusion in a weekly dosage of from 10 to 1500 mg/m², such as from 30 to 1500 mg/m², or such as from 50 to 1000 mg/m², or such as from 10 to 500 mg/m², or such as from 100 to 300mg/m². Such administration may be repeated, e.g., 1 time to 8 times, such as 3 times to 5 times. The administration may be performed by continuous infusion over a period of from 2 hours to 24 hours, such as from 2 hours to 12 hours.

In a thirtieth embodiment, the anti-HERV-K humanized antibodies or ADCs may be administered by infusion every third week in a dosage of from 30 to 1500 mg/m², such as from 50 to 1000 mg/m² or 100 to 300 mg/m². Such administration may be repeated, e.g., 1 time to 8 times, such as 3 times to 5 times. The administration may be performed by continuous infusion over a period of from 2 hours to 24 hours, such as from 2 hours to 12 hours.

In a thirty-first embodiment, the anti-HERV-K humanized antibodies or ADCs may be administered by slow continuous infusion over a prolonged period, such as more than 24 hours, to reduce toxic side effects.

In a thirty-second embodiment the anti-HERV-K humanized antibodies or ADCs may be administered in a weekly dosage of 50 mg to 2000 mg, such as for example 50 mg, 100 mg, 200 mg, 300 mg, 500 mg, 700 mg, 1000 mg, 1500 mg, or 2000 mg, for up to 16 times, such as from 4 to 10 times, such as from 4 to 6 times. The administration may be performed by continuous infusion over a period from 2 to 24 hours, such as from 2 to 12 hours. Such regimen may be repeated one or more times as necessary, for example, after 6 months or 12 months. The dosage may be determined or adjusted by measuring the amount of anti-HERV-K humanized antibodies or ADCs of the invention in the blood upon administration, by for instance taking out a biological sample and using anti-idiotypic antibodies which target the antigen binding region of the anti-HERV-K humanized antibodies or ADCs of the invention.

In a thirty-third embodiment, the anti-HERV-K humanized antibodies or ADCs may be administered by maintenance therapy, such as, e.g., once a week for a period of 6 months or more.

In a thirty-fourth embodiment, the ADC may be administered by a regimen including one infusion of an ADC of the invention followed by an infusion of an anti-HERV-K antibody of the invention, such as antibody 6H5hum.

### Bispecific T Cell Engagers (BiTEs)

In a thirty-fifth embodiment, provided herein is a method of treating a HERV-K-positive cancer in a subject in need thereof, comprising administering to the subject a therapeutically effective amount of a bispecific antibody comprising two different antigen-binding regions, one which has a binding specificity for CD3 or CD8 and one which has a binding specificity for HERV-K.

In a thirty-sixth embodiment, the invention relates to a bispecific antibody comprising a first single chain human variable region which binds to HERV-K, in series with a second single chain human variable region which binds to T cell activation ligand CD3 or CD8. T the first and second single chain human variable regions are in amino to carboxy order, wherein a linker sequence intervenes between each of said segments, and wherein a spacer polypeptide links the first and second single chain variable regions.

In a thirty-seventh embodiment of the method, the administering is intravenous or intraperitoneal.

In a thirty-eighth embodiment of the method, the bispecific binding molecule is not bound to a T cell during said administering step.

In a thirty-ninth embodiment of a method described herein, the method further comprises administering T cells to the subject. In a specific embodiment, the T cells are bound to molecules identical to said bispecific binding molecule.

In a fortieth embodiment, provided herein is a pharmaceutical composition comprising a therapeutically effective amount of the bispecific binding molecule, a pharmaceutically acceptable carrier, and T cells. In a forty-first embodiment, the T cells are bound to the bispecific binding molecule. In a forty-second embodiment, the binding of the T cells to the bispecific binding molecule is noncovalently. In a forty-third embodiment, the administering is performed in combination with T cell infusion to a subject for treatment of a HERV-K-positive cancer. In a forty-fourth embodiment, the administering is performed after treating the patient with T cell infusion. In a forty-fifth embodiment, the T cells are autologous to the subject to whom they are administered. In a forty-sixth embodiment, the T cells are allogeneic to the subject to whom they are administered. In a forty-seventh embodiment, the T cells are human T cells.

In a forty-eighth embodiment of a method described herein, the subject is a human.

In a forty-ninth embodiment of the method, the bispecific binding molecule is contained in a pharmaceutical composition, which pharmaceutical composition further comprises a pharmaceutically acceptable carrier.

In a fiftieth embodiment of the bispecific binding molecule, the bispecific binding molecule does not bind an Fe receptor in its soluble or cell-bound form. In some embodiments of the bispecific binding molecule, the heavy chain was mutated to destroy an N-linked glycosylation site. In a fifty-first embodiment of the bispecific binding molecule, the heavy chain has an amino acid substitution to replace an asparagine that is an N-linked glycosylation site, with an amino acid that does not function as a glycosylation site. In a fifty-second embodiment of the bispecific binding molecule, the heavy chain was mutated to destroy a C1q binding site. In a fifty-third embodiment, the bispecific binding molecule does not activate complement.

In a fifty-fourth embodiment of the bispecific binding molecule, the HERV-K-positive cancer is breast cancer, ovarian cancer, prostate cancer, pancreatic cancer, melanoma, colorectal cancer, small cell lung cancer, non-small cell lung cancer or any other neoplastic tissue that expresses HERV-K. In a fifty-fifth embodiment, the HERV-K-positive cancer is a primary tumor or a metastatic tumor, e.g., brain, bone, or lung metastases.

### DNA-Encoded Hi-Specific T Cell Engagers (DBiTE)

Specific antibody therapy, including mAbs and bispecific T cell engagers (BiTEs), are important tools for cancer immunotherapy. BiTEs are a class of artificial bi-specific monoclonal antibodies that has the potential to transform the immunotherapy landscape for cancer. BiTEs direct a host's immune system, more specifically the T cells' cytotoxic activity, against cancer cells. BiTEs have two binding domains. One domain hinds to the targeted tumor (like HERV-K-expressing cells) while the other engages the immune system by binding directly to molecules on T cells. This double-binding activity drives T cell activation directly at the tumor resulting in a killing function and tumor destruction. DBiTEs share many advantages of bi-specific monoclonal antibodies. Both are composed of engineered DNA sequences which encode two antibody fragments. The patient's own cells become the factory to manufacture functional BiTES encoded by the delivered DBiTE sequences. Delivery of BiTEs and permitting combinations of DBiTEs to be administered at one time as a multi-pronged approach to treat resistant cancer. Synthetic DNA designs for BiTE-like molecules include engineering and encoding them in optimized synthetic plasmid DNA cassettes. DBiTEs are then injected locally into the muscle and muscle cells convert the genetic instructions into protein to allow for direct in vivo launching of the molecule directly into the bloodstream to the seek and destroy tumors. See, Perales-Puchalt et al., DNA-encoded bispecific T cell engagers and antibodies present long-term antitumor activity, JCI Insight, 4(8), e126086 (Apr. 18, 2019). In preclinical studies, DBiTEs demonstrated a unique profile compared to conventional BiTEs, overcoming some of the technical challenges associated with production. For further information, see also, PCT Pat. Publ. WO 2016/054153 (The Wistar Institute of Anatomy and Biology) and WO 2018/041827 (Psioxus Therapeutics Limited).

### HERV-K CAR-T Therapies

Many formulations of CARs specific for target antigens have been developed. See e.g., International Pat. Publ. WO 2014/186469 (Board of Regents, the University of Texas System). This specification provides a method of generating chimeric antigen receptor (CAR)-modified T cells with long-lived in vivo potential for the purpose of treating, for example, leukemia patients exhibiting minimal residual disease (MRD). In aggregate, this method describes how soluble molecules such as cytokines can be fused to the cell surface to augment therapeutic potential. The core of this method relies on co-modifying CART cells with a human cytokine mutein of interleukin-15 (IL-15), henceforth referred to as mIL15. The mIL15 fusion protein is comprised of codon-optimized cDNA sequence of IL-15 fused to the full length IL15 receptor alpha via a flexible serine-glycine linker. This IL-15 mutein was designed in such a fashion so as to: (i) restrict the mIL15 expression to the surface of the CAR+ T cells to limit diffusion of the cytokine to non-target in vivo environments, thereby potentially improving its safety profile as exogenous soluble cytokine administration has led to toxicities; and (ii) present IL-15 in the context of IL-15Ra to mimic physiologically relevant and qualitative signaling as well as stabilization and recycling of the IL15/IL15Ra complex for a longer cytokine half-life. T cells expressing mIL15 are capable of continued supportive cytokine signaling, which is critical to their survival post-infusion. The mIL15+CAR+ T cells generated by non-viral Sleeping Beauty System genetic modification and subsequent ex vivo expansion on a clinically applicable platform in yielded a T cell infusion product with enhanced persistence after infusion in murine models with high, low, or no tumor burden. Moreover, the mIL15 CAR+ T cells also demonstrated improved anti-tumor efficacy in both the high and low tumor burden models. A hu6H5 scFv was used to generate a K-CAR in a lentiviral vector.

### Combination Therapies

The therapies of this specification can be used without modification, relying on the binding of the antibodies or fragments to the surface antigens of HERV-K+ cancer cells in situ to stimulate an immune attack thereon. Alternatively, the aforementioned method can be carried out using the antibodies or binding fragments to which a cytotoxic agent is bound. Binding of the cytotoxic antibodies, or antibody binding fragments, to the tumor cells inhibits the growth of or kills the cells.

Antibodies specific for HERV-K env protein may be used in conjunction with other expressed HERV antigens. This may be particularly useful for immunotherapy and antibody treatments of diseases in which several different HERVs are expressed. For example, HERV-E in prostate, ERV3, HERV-E and HERV-K in ovarian cancer, and ERV3, HERV-H, and HERV-W in other cancers.

Cytokines in the common gamma chain receptor family (γC) are important costimulatory molecules for T cells that are critical to lymphoid function, survival, and proliferation. IL-15 possesses several attributes that are desirable for adoptive therapy. IL-15 is a homeostatic cytokine that supports the survival of long-lived memory cytotoxic T cells, promotes the eradication of established tumors via alleviating functional suppression of tumor-resident cells, and inhibits activation-induced cell death (AICD). IL-15 is tissue restricted and only under pathologic conditions is it observed at any level in the serum, or systemically. Unlike other γC cytokines that are secreted into the surrounding milieu, IL-15 is trans-presented by the producing cell to T cells in the context of IL-15 receptor alpha (IL-15Ra). The unique delivery mechanism of this cytokine to T cells and other responding cells: (i) is highly targeted and localized, (ii) increases the stability and half-life of IL-15, and (iii) yields qualitatively different signaling than is achieved by soluble IL-15.

### Pharmaceutical Compositions

This specification is also directed to pharmaceutical compositions comprising a therapy that specifically binds to a HERV-K env protein, together with a pharmaceutically acceptable carrier, excipient, or diluent. Such pharmaceutical compositions may be administered in any suitable manner, including parental, topical, oral, or local (such as aerosol or transdermal) or any combination thereof. Suitable regimens also include an initial administration by intravenous bolus injection followed by repeated doses at one or more intervals.

Pharmaceutical compositions of the compounds of the disclosure are prepared for storage by mixing a peptide ligand containing compound having the desired degree of purity with optional pharmaceutically acceptable carriers, excipients, or stabilizers (Remington's Pharmaceutical Sciences 18th ed., 1990), in the form of lyophilized formulations or aqueous solutions. Acceptable carriers, excipients, or stabilizers are nontoxic to recipients at the dosages and concentrations used.

The compositions herein may also contain more than one active compound as necessary for the particular indication being treated, preferably those with complementary activities that do not adversely affect each other. Alternatively, or in addition, the composition may comprise a cytotoxic agent, cytokine, growth inhibitory agent and/or cardioprotectant, Such molecules are suitably present in combination in amounts that are effective for the purpose intended.

The invention is further illustrated by the following examples, which are not intended to limit the scope or content of the invention in any way.

### EXAMPLES

### Example 1

### Generation of hu6H5 Ab by CDR Grafting

VL
(SEQ ID NO: 4)
GACATCGAGCTCACTCAGTCTCCAGCTTCTTTGGCTGTGTCTCTAGGGC
AGAGGGCCACCATATCCTGCAGAGCCAGTGAAAGTGTTGATAGTCATGG
CACTAGTTTTATGCACTGGTACCAGCAGAAACCAGGACAGCCACCCAAA
TTCCTCATCTATCGTGCATCCAACCTAGAATCTGGGATCCCTGCCAGGT
TCAGTGGCAGTGGGTCTAGGACAGACTTCACCCTCACCATTAATCCTGT
GGAGACAGATGATGTTGCAATCTATTACTGTCAGCAAAGTAATGAGGAT
CCTCCGACGTTCGGTGGAGGCACCAAGCTGGAAATCAAAC
>FWJ_VL
(SEQ ID NO: 5)
DIELTQSPASLAVSLGQRATISCRASESVDSHGTSFMHWYQQKPGQPPK
FLIYRASNLESGIPARFSGSGSRIDFTLTINPVETDDVAIYYCQQSNED
PPTFGGGIKLEIK

| | | |
|---|---|---|
| 1 | D I E L T Q S P A S L A V S L G Q R A T | 20 |
| 1 | GACATCGAGCTCACTCAGTCTCCAGCTTCTTTGGCTGTGTCTCTAGGGCAGAGGGCCACC | 60 |
| 21 | I S C R A S E S V D S H G T S F M H W Y | 40 |
| 61 | ATATCCTGCAGAGCCAGTGAAAGTGTTGATAGTCATGGCACTAGTTTTATGCACTGGTAC | 120 |
| 41 | Q Q K P G Q P P K F L I Y R A S N L E S | 60 |
| 121 | CAGCAGAAACCAGGACAGCCACCCAAATTCCTCATCTATCGTGCATCCAACCTAGAATCT | 180 |
| 61 | G I P A R F S G S G S R T D F T L T I N | 80 |
| 181 | GGGATCCCTGCCAGGTTCAGTGGCAGTGGGTCTAGGACAGACTTCACCCTCACCATTAAT | 240 |
| 81 | P V E T D D V A I Y Y C Q Q S N E D P P | 100 |
| 241 | CCTGTGGAGACAGATGATGTTGCAATCTATTACTGTCAGCAAAGTAATGAGGATCCTCCG | 300 |
| 101 | T F G G G T K L E I K | 111 (SEQ ID NO: 5) |
| 301 | ACGTTCGGTGGAGGCACCAAGCTGGAAATCAAA | 333 (SEQ ID NO: 4) |

### Design of Humanized Single Chain Variable Fragment (scFv) Antibody

Antibody numbering scheme and CDR definitions: The antibody-numbering server is part of KabatMan database (http://www.bioinf.org.uk/) and was used to number all antibody sequences of this study according to the enhanced Chothia scheme. In this humanization study, the inventors have combined the Enhanced Chothia numbering with the Contact CDR definition of antibody sequence to position the CDRs of antibody light chain and heavy chains at the following locations: H-CDR1 30-35, H-CDR2 47-58 H-CDR3 93-101, L-CDR1 30-36, L-CDR2 46-55, and L-CDR3 89-96.

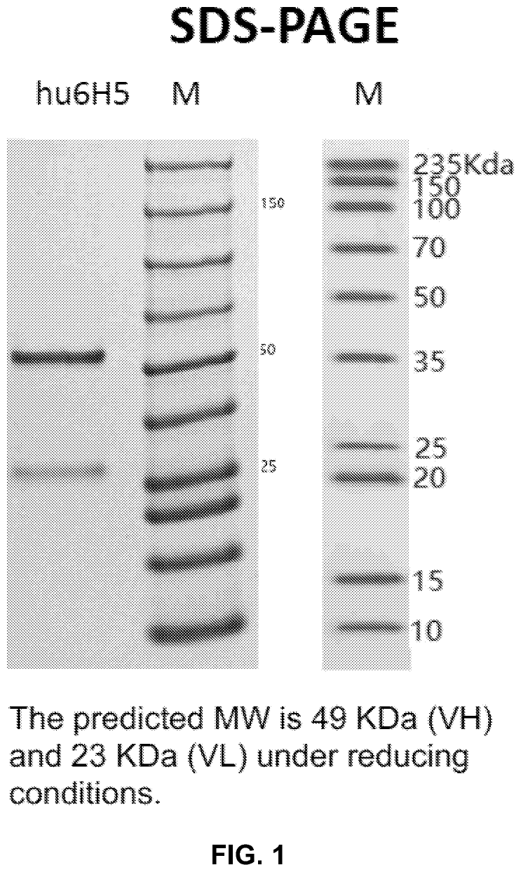

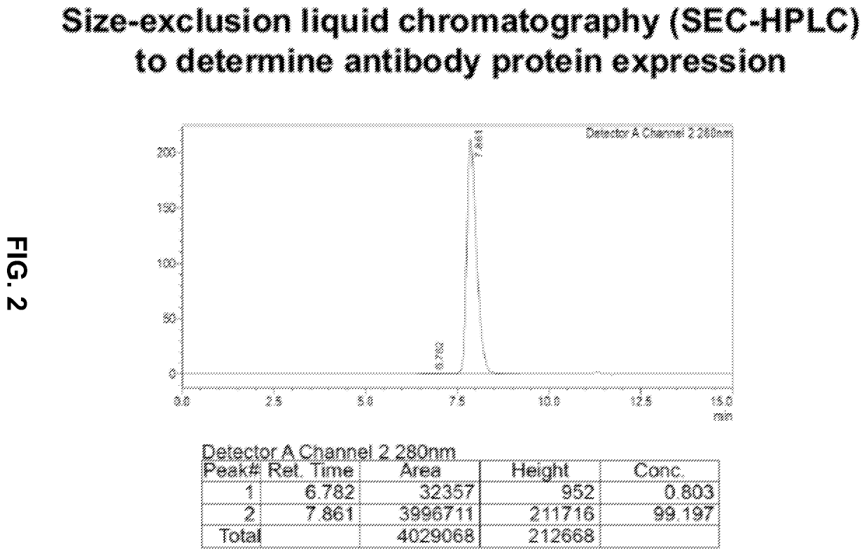

Selection of the human template: To generate humanized scFv gene, six Complementary determine regions (CDRs) of mouse VH and VL were grafted onto selected human Frameworks (FRs) showing the highest amino acids sequence identify to the humanization of the given antibodies. The human immunoglobulin germline sequences were used as the selected human FRs for mouse FWJ antibody clone (FIG. 1). Human immunoglobulin germline sequences showing the highest amino acid sequences similarity in FRs between human and mouse FWJ VH and VL were identified independently using from V-quest server (http://www.imgt.org/IMGT_vquest) and Ig-BLAST server (http://www.ncbi.nlm.nih.gov/igblast). The selected heavy chain VHIII and the light KJ chain based conserved germlines. The consensus human FRs was designed among selected germline gene for grafting CDRs residues of FWJ. The amino acid sequences in FRs of mouse VH and VL that differed from consensus human FRs were substituted with human residues, while preserving mouse residues at position known as Vernier zone residues and chain packing residues.

>HUM1-FWJVH
(SEQ ID NO: 6)
EVQLVESGGGLVQPGGSLRLSCKASGYSFTGYYMHWVRQAPGKGLEWIGRVNPNSGGTSY
NQKFKDRATLSVDNSKNTAYLQMNSLRAEDTAVYYCARSKGNYFYAMDYWGQGTLVTVSS.
The Z-score of the query sequence is: 0.7.
>Hum2 FWJVH VH
(SEQ ID NO: 7)
EVQLVESGGGLVQPGGSLKVSCKASGYSFTGYYMHWVRQASGKGLEWIGRVNPNSGGTSY
NQKFKDRFTISRDKSISTLYLOMSSLRSEDTAVYYCARSKGNYFYAMDYWGQGTLVTVSS.
The Z-score of the query sequence is: 0.5.
>FWJ_VH
(SEQ ID NO: 8)
QVKLQQSGPDLVKPGASVKISCKASGYSFTGYYMHWVKQSHGKSLEWIGRVNPNSGGTSY
NOKFKDKAILTVDKSSSTAYMELRSLTSEDSAVYYCARSKGNYFYAMDYWGQGTTVTVSS.
The Z-score of the query sequence is: −1.7.

TABLE 4

| | Z value | Z value |
|---|---|---|
| | (humanness of | (humanness of |
| Gene sequence | VH ) | VL ) |
| Mouse Gene (FWJ) | −1.7 | −1.0 |
| Humanized version 1(Hum1-FWJ) | 0.7 | 0.1 |
| Humanized version 2(Hum2-FWJ) | 0.5 | 0.0 |

>HUM1FWJVL
(SEQ ID NO: 9)
DIQMTQSPSSLSASVGDRVTITCRASESVDSHGTSFMHWYQQKPGKAPKFLIYRASNLES
GIPSRFSGSGSGTDFTLTISSVQPEDFAVYYCQQSNEDPPTFGGGTKVEIK.
The Z-score of the query sequence is: 0.1.
>HUM2FWJVL
(SEQ ID NO: 10)
DIQMTQSPSSLSASVGDRVTISCRASESVDSHGTSFMHWYQQKPGKSPKFLIYRASNLES
GIPSRFSGSGSGTDFTLTISSLQPEDFAIYYCQQSNEDPPTFGGGTKVEIK.
The Z-score of the query sequence is: 0.0.
>FWJ_VL
(SEQ ID NO: 11)
DIELTQSPASLAVSLGQRATISCRASESVDSHGTSFMHWYQQKPGQPPKFLIYRASNLES
GIPARFSGSGSRIDFTLTINPVETDDVAIYYCQQSNEDPPTFGGGTKLEIK.
The Z-score of the query sequence is: −1.0.
Final humanized version of scFv gene.
Humanized scFv-1
(SEQ ID NO: 12)
EVQLVESGGGLVQPGGSLRLSCKASGYSFTGYYMHWVRQAPGKGLEWIGRVNPNSGGTSY
NQKFKDRATLSVDNSKNTAYLQMNSLRAEDTAVYYCARSKGNYFYAMDYWGQGTLVTVSS
GGGGSGGGGSGGGGSDIQMTQSPSSLSASVGDRVTITCRASESVDSHGTSFMHWYQQKPG
KAPKFLIYRASNLESGIPSRFSGSGSGTDFTLTISSVQPEDFAVYYCQQSNEDPPTFGGG
TKVEIK
Humanized scFv-2
(SEQ ID NO: 13)
EVQLVESGGGLVQPGGSLKVSCKASGYSFTGYYMHWVRQASGKGLEWIGRVNPNSGGTSY
NQKFKDRFTISRDKSISTLYLQMSSLRSEDTAVYYCARSKGNYFYAMDYWGQGTLVTVSS
GGGGSGGGGSGGGGSDIQMTQSPSSLSASVGDRVTISCRASESVDSHGTSFMHWYQQKPG
KSPKFLIYRASNLESGIPSRFSGSGSGTDFTLTISSLQPEDFAIYYCQQSNEDPPTEGGG
TKVEIK

Construction of scFv and test of biological activity against Human KV and 231 antigens. The clone of variable heavy chain and light chain of FWJ_1 and FWJ_2 antibody gene were amplified and synthesized. The gene encoding the scFv is VH-linker-VL with a standard 20 amino acid linker (Gly4Ser) 3 GGGAR (SEQ ID NO: 14). The amplified gene was digested with BssHII and NheI restriction enzymes and insert into a pET-based vector (PAB-myc) containing a pelB promotor for controlling periplasmic protein expression (Novagen, Madison, WI, USA) along with 6xhistidine tag at the C-termini for purification by metal affinity chromatography and transformed into DH5α bacterial strain. The transformed clones were amplified in LB with ampicillin broth overnight. The plasmid DNAs were prepared and sent for DNA sequencing. The correct sequence of scFv plasmid was transformed into the T7 Shuffle bacterial strain and the transformed bacteria were used for soluble protein production in periplasmic compartment.

FWJ_1 and FWJ_2_scFv_Gene and Translated Protein Sequences: The diagram below delineates the Heavy and Light Chains and Linker Arm of FWJ_1 and FWJ_2_scFv. In the engineering of the FWJ_1 and FWJ_2_scFv gene two epitope tags were engineered onto the C terminus: 1) a 6 his tag to facilitate purification of the encoded scFv by nickel affinity chromatography; and 2) a myc tag to facilitate rapid immunochemical recognition of the expressed scFv.

Induction of ScFv proteins in a bacterial host: The FWJ_1 and FWJ_2scFv clone were transformed into T7 shuffle bacterial strain. T7 shuffle cells and was grown in 1.4 L 2×YT plus ampicillin medium at 37° C. until log-phage (OD600=0.5), induced with 0.3 mM IPTG, and allowed to grow at 30° C. for an additional 16 h. After induction, the bacteria were harvested by centrifugation at 8000 g for 15 min at 4° C., and the pellets were stored in −20° C. for at least 2 hours. The frozen pellets were briefly thawed and suspended in 40 ml of lysis buffer (1 mg/ml lysozyme in PBS plus EDTA-free protease inhibitor cocktail (Thermo Scientific, Waltham, MA, USA). The lysis mixture was incubated on ice for an hour, and then 10 mM MgCL₂ and 1 μg/ml DNaseI were added, and the mixture was incubated at 25° C. for 20 min. The final lysis mixture was centrifuged at 12000 g for 20 minutes and the supernatants were collected. This supernatant was termed the periplasmic extract used for nickel column affinity chromatography.

Western blot analysis using FWJ_1 and FWJ_2 scFv protein: Lysate Ag and KSU protein were used as antigens target in Dot-blot analyses. 2-5 ug Ag proteins as non-reduced conditional and lug purified protein as negative control were loaded onto nitrocellulose membranes. The membrane was blocked using 3% skimmed milk in PBS for 3 h at room temperature. After that, the membrane was incubated with periplasmic extract of FWJ_1 and FWJ_2 scFv proteins overnight at 4° C. The membrane was washed with sodium phosphate buffered saline with 0.05% tween 20 buffer (PBST) 3 times. The washed membrane was incubated with anti-c Myc mouse IgG for 1 h at room temperature to recognize the c-Myc tag on the scFv and identify the position of antigens bound by the scFv. After washing with PBST, the membrane was incubated with the goat anti-mouse IgG (H+L) HRP conjugate diluted (1:3000 v/v) in PBS for 1 h at RT, and specific immunoreactive bands were visualized with a mixture of TMB substrate.

The inventors identified anti-HERV-K mAb 6H5 heavy chain CDRs (H-CDR1 30-35, H-CDR2 47-58, H-CDR3 93-101), and light chain CDRs (L-CDR1 30-36, L-CDR2 46-55, and L-CDR3 89-96) and grafted them onto selected human frameworks (FRs) showing the highest amino acid sequence identity to optimize the humanization of the given antibodies. Human immunoglobulin germline sequences showing the highest amino acid sequence similarity in FRs between human and mouse VH and VL were identified independently from the V-quest (http://www.imgt.org/IMGT_vquest) and Ig-BLAST (http://www.ncbi.nlm.nih.gov/igblast) servers. The amino acid sequences in FRs of mouse VH and VL that differ from consensus human FRs were substituted with human residues, while preserving mouse residues at positions known as Vernier zone residues and chain packing residues. The clone of VH and VL chains of candidate humanized antibody genes were amplified and synthesized. The gene encoding the scFv, which includes a VH-linker-VL with a standard 20 amino acid linker (Gly4Ser) 3 GGGAR, was inserted into a pET based vector (PAB-myc) containing a pelB promotor for controlling periplasmic protein expression (Novagen, Madison, WI) along with a 6x histidine tag at the C-termini for purification by metal affinity chromatography and a myc tag to facilitate rapid immunochemical recognition of the expressed scFv. The correct sequences of the scFv plasmid were used for soluble protein production in the periplasmic compartment. Two hu6H5 clones (FWJ1 and FWJ2) were selected and binding affinities to antigen were determined. Both clones were able to bind antigens produced from recombinant HERV-K Env surface fusion protein (KSU) and lysates from MDA-MB-231 breast cancer cells.

HuVH or HuVL with human IgG1 was cloned into a pcDNA 3.4 vector to produce VH-CH (human IgG1) or VL-CL (human Kappa). The plasmids were transiently transfected into Expi293 cells for mammalian expression. The ratio of H chain vs. L chain plasmids is 2:3. A Western Blot was used to determine expression, and the predicted MW of 49/23 kDa (H chain/L chain) under reducing conditions was detected (FIG. 1).

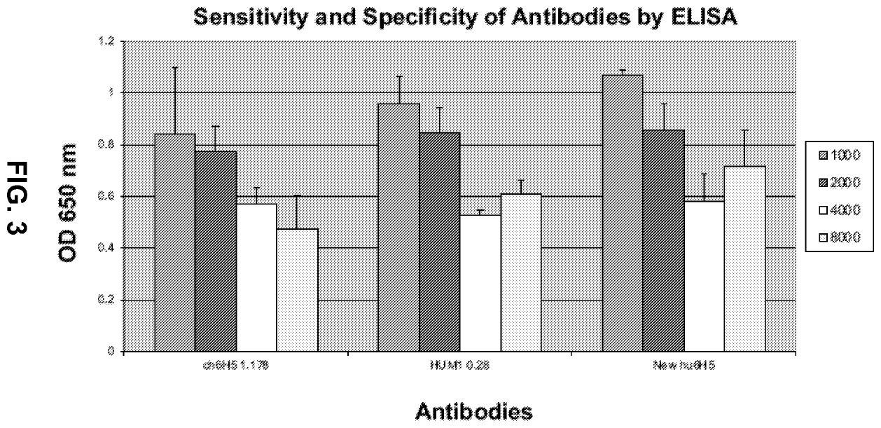

Size-exclusion chromatography (SEC) separation by size and/or molecular weight was further employed to determine protein expression (FIG. 2). Finally, the humanized 6H5 antibody (Purity>95%) with an endotoxin level<1 EU/mg was used to determine antitumor effects in vitro and in vivo.

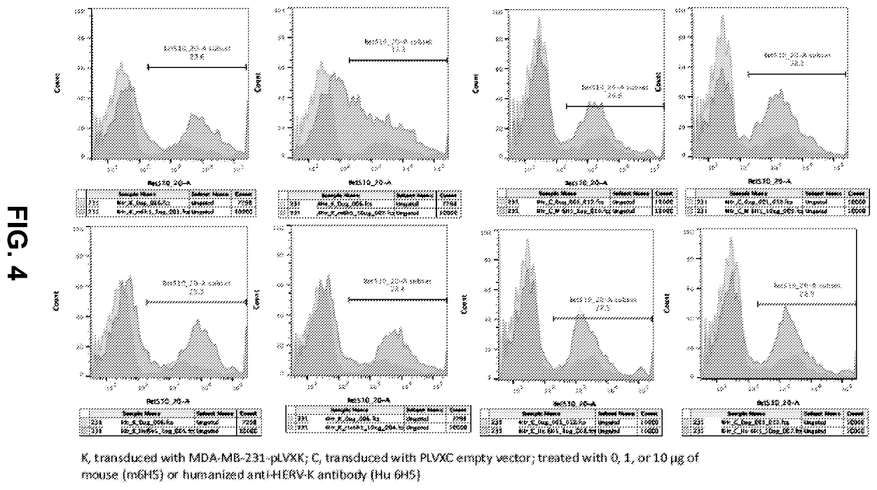

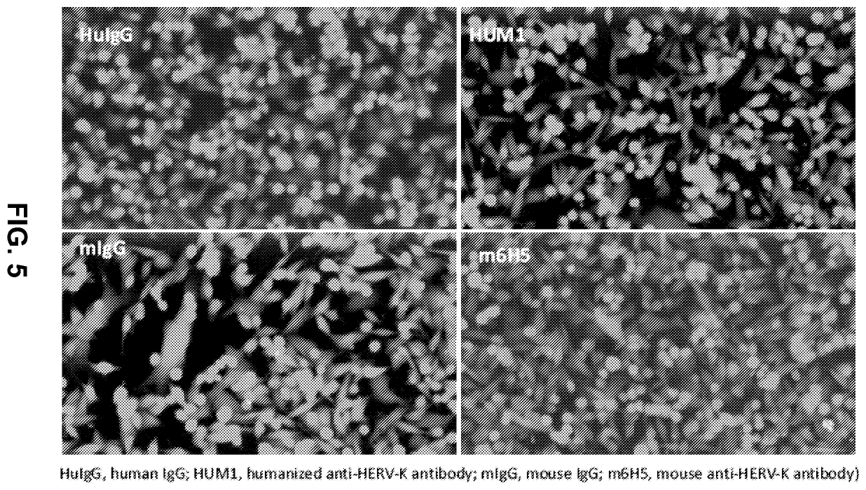

An ELISA assay was employed to compare antigen binding sensitivity and specificity of hu6H5 vs. m6H5 (FIG. 3). No significant difference between these two parameters was detected.

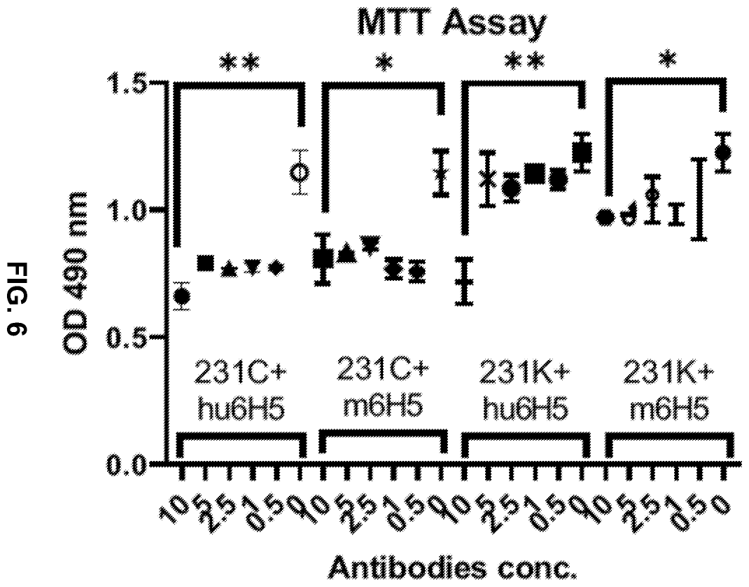

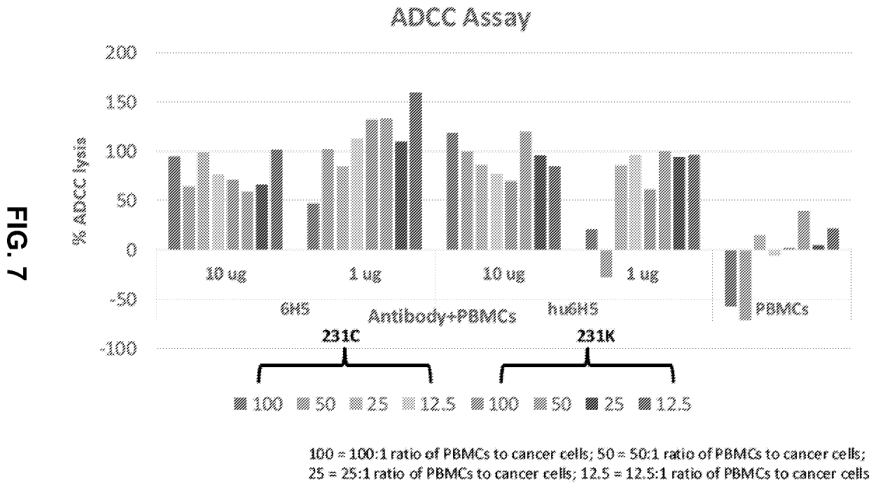

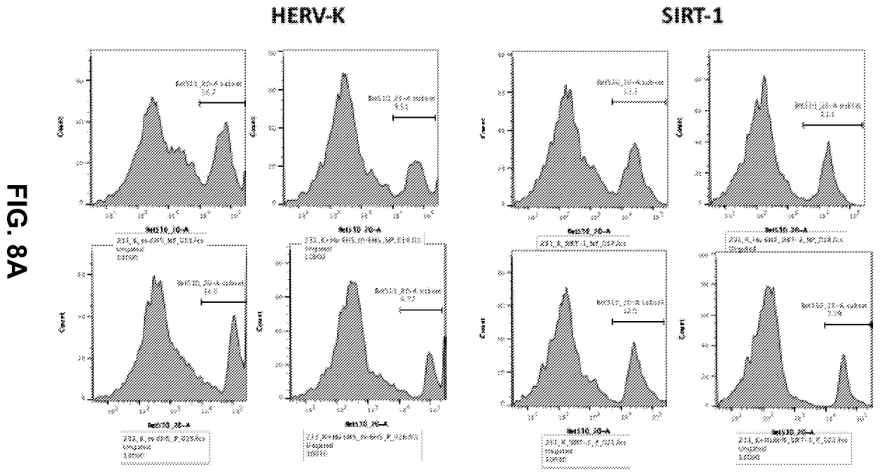

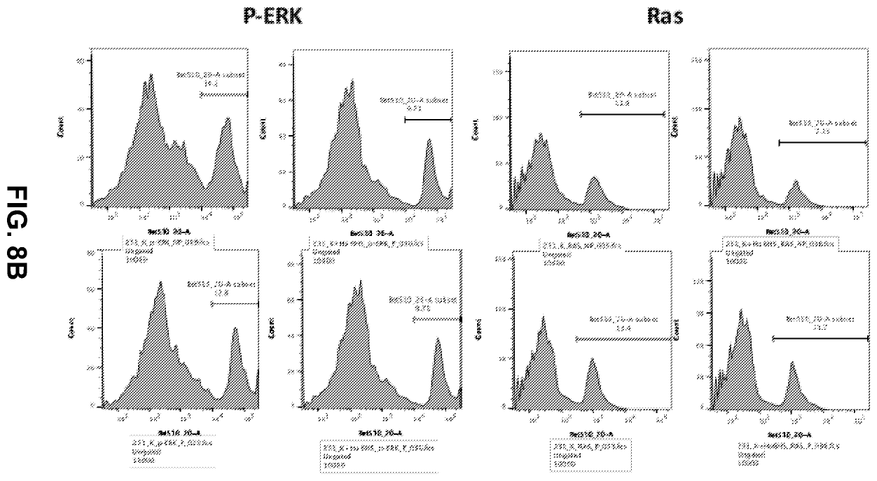

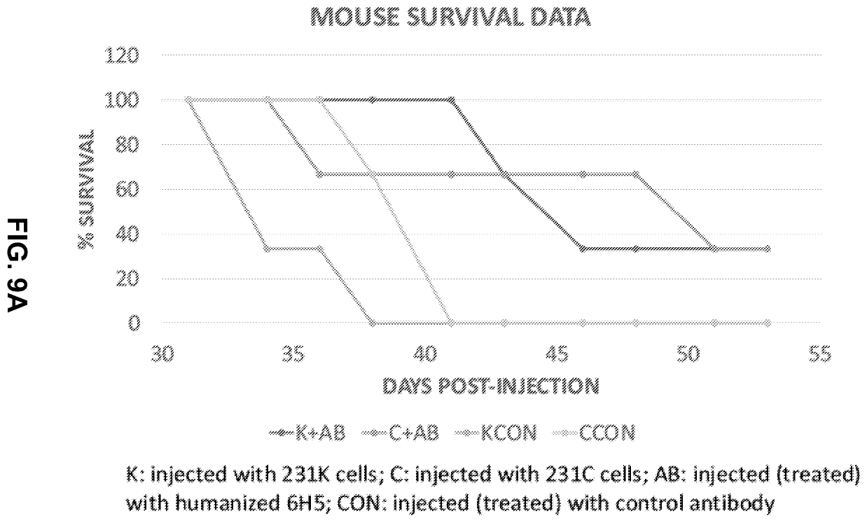

An apoptosis assay was used to compare the efficacy of hu6H5 and m6H5 in killing cancer cells. The respective antibodies (1 or 10 ug per ml) were used to treat MDA-MB-231 breast cancer cells for 4 hours and 24 hours (FIG. 4). Cells treated with no antibody or with mIgG or human IgG were used as control. The results showed that hu6H5 had a similar effect as m6H5 at killing these breast cancer cells. To further evaluate efficacy in cell killing, MDA-MB-231 cells were treated with various antibodies (10 ng/ml) for 16 hr. Live cells (green color; Calcein Am) and putative dead cells (red color; EthD-1) were identified using the co-stained Live/Dead Viability Assay (FIG. 5), and the results showed that hu6H5 had an effectiveness that is similar to m6H5 at killing breast cancer cells. Furthermore, an MTT (3-(4,5-Dimethylthiazol-2-yl)-2,5-Diphenyltetrazolium Bromide) assay was employed to confirm that hu6H5 can inhibit cancer cell growth (FIG. 6). ADCC was used to determine the mechanism of BC cell killing, and our results support effector cell mediated secretion of cytotoxic molecules that lyse antibody-coated target cells (FIG. 7).

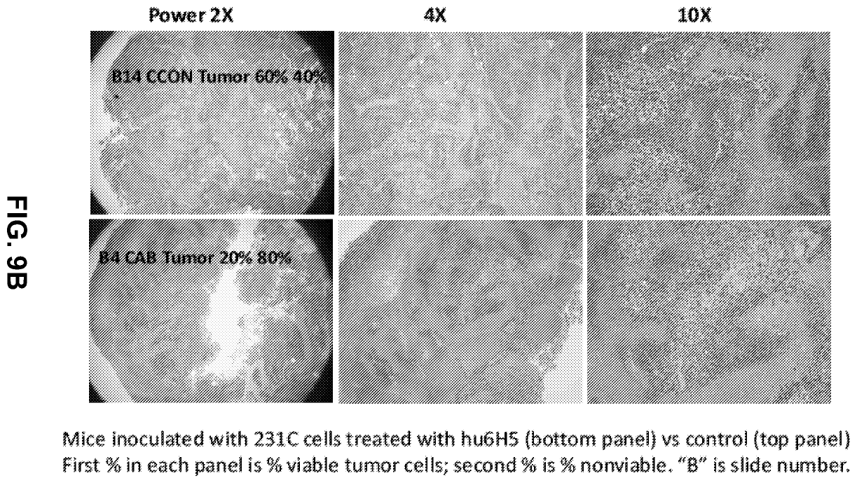

Flow cytometry was employed to determine if hu6H5 can downregulated the expression of p-ERK, Ras, and SIRT-1. 231 C or 231 K cells were treated with 10 μg per ml of hu6H5 for 16 hr. The expression of HERV-K, SIRT-1 (FIG. 8A), p-ERK and Ras (FIG. 8B) in both cells under perm and non-perm. Down-regulated expression of HERV-K, p-ERK, Ras, and SIRT-1 was demonstrated in 231 K treated with hu6H5 or 231C.

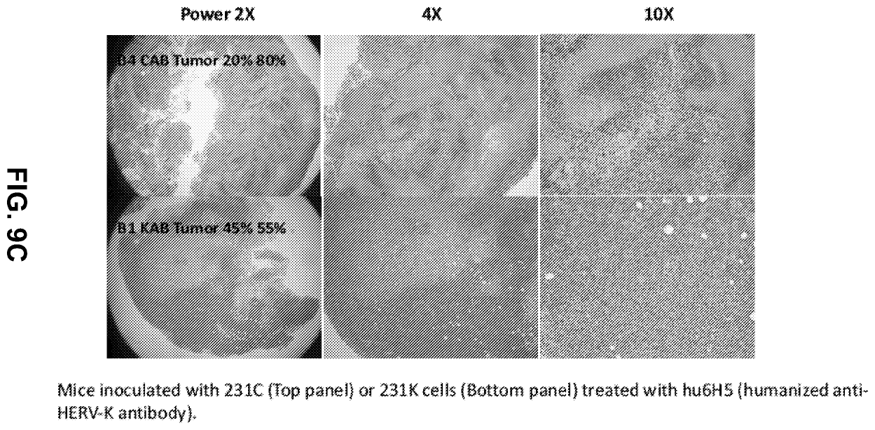

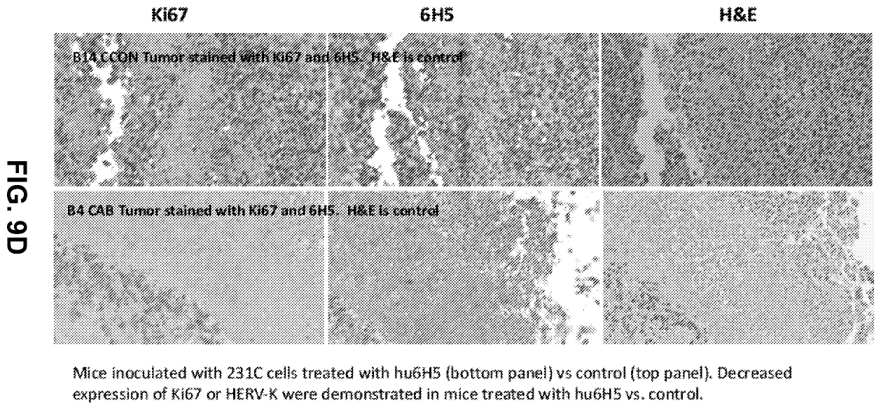

pLVXK is an HERV-K expression vector, and MDA-MB-231 pLVXK are MDA-MB-231 cells that were transduced with pLVXK. Likewise, pLVXC is control expression vector only, and MDA-MB-231 pLVXK are MDA-MB-231 cells that were transduced with pLVXC. NSG female mice (8-week-old), were inoculated with MDA-MB-231 pLVXC (231-C; subcutaneous, 2 million cells) vs, MDA_MB-231 pLVXK (231-K; subcutaneous, 2 million cells). On day 6, mice were treated with hu6H5 (4 mg/kg intraperitoneal, twice weekly for 3 weeks). Tumor growth was monitored and measured every other day. The percentage of mice surviving at various time intervals is shown in FIG. 9A. Higher survival was demonstrated in mice bearing 231-C and 231-K cells treated with antibodies. Tumor and lung tissues were collected from each mouse. Larger lymph nodes were detected in some mice bearing 231-K cells but not in mice bearing 231-C cells.

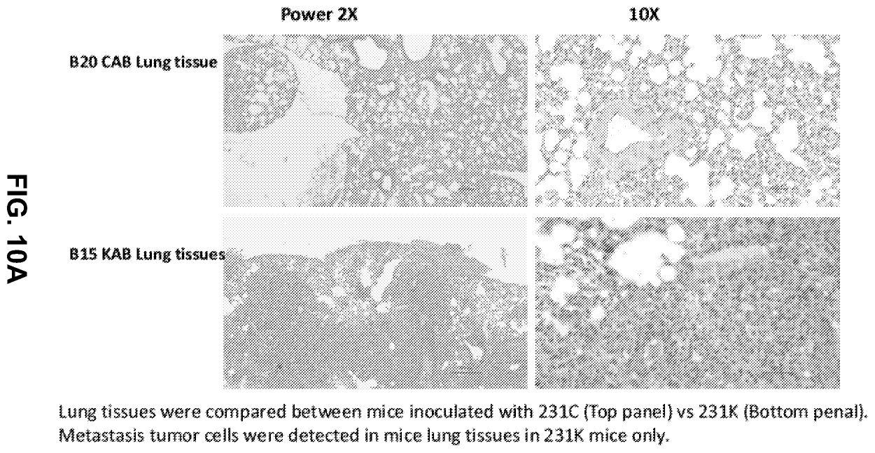

Hematoxylin and eosin (H&E) staining was further used to assess morphological features of tumor tissues (FIG. 9) and tissues from other organs (lungs and lymph nodes; FIG. 10). Tumor viability and tumor necrosis were quantitated by a pathologist by measuring the tumor areas by H&E staining. Humanized antibody treatment resulted in smaller tumor volumes, less tumor focality and number, less infiltrative borders, and decreased mitotic activities. A reduced percentage of tumor viability was observed in mice bearing 231-C cells (FIG. 9B) or in 231-K cells (FIG. 10B) treated with antibody. Reduced tumor variability was demonstrated in 231C (FIG. 9B) or 231K cells (FIG. 9C) treated with hu6H5, relative to their controls. Anti-Ki67 and anti-HERV-K mAb were used (FIG. 9D). Reduced tumor viability was demonstrated in mice treated with hu6H5 (20%; bottom panel) compared with control (60%; top panel; FIG. 9B). The antibody treatment groups were more uniform in appearance, with less pleomorphic nuclei and smaller nucleoli, and tumor-infiltrating lymphocytes were significantly increased in number.

Metastatic tumor cells were also found in lung tissues obtained from mice bearing 231-K cells, but not in mice bearing 231-C cells (FIG. 10A). Reduced percentages of tumor viability were observed in lungs of mice bearing 231-K treated with antibody compared to those not treated with antibody (FIG. 10B). Metastatic lymph nodes were detected only in mice inoculated with 231K cells (FIG. 10C). Greater than 95% tumor viability was detected in lymph nodes in price bearing 231-K cells (FIG. 10C; >95%; top panel) and reduced percentages of tumor viability were observed in lymph nodes in mice treated with antibody (FIG. 10C; 40%; bottom panel) compared to mice not treated with antibody (FIG. 10D). Ascites were demonstrated in mice that carried 231K or 231 C tumor cells without antibody treatment.

### Example 2

### Efficacy of a Bispecific T Cell Engager (BiTE) Targeting HERV-K

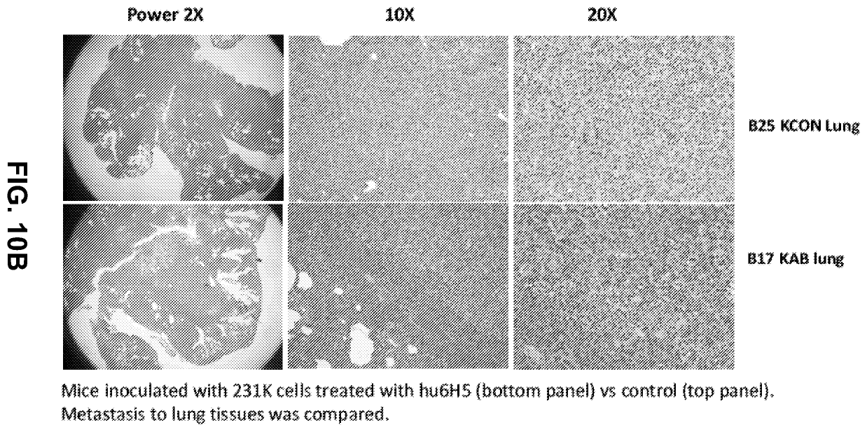

A BiTE directed against T cell CD3 or CD8 and the tumor-associated antigen HERV-K was produced, comprised of antibodies targeting either CD3 or CD8 and HERV-K. This BiTE was shown to elicit interferon-gamma (IFN gamma) cytotoxic activity towards MDA-MB-231 breast cancer cells expressing major histocompatibility class (MHC) molecules loaded with HERV-K epitopes, with 20-30-fold increases in IFN gamma expression after treatment with the BiTE (FIG. 11).

A BiTE is a recombinant protein built as a single-chain antibody construct that redirects T cells to tumor cells, and that does not require expansion of endogenous T cells through antigen-presenting cells. See scientific reference 50. BiTE molecules can be administered directly to patients and BiTE-mediated T cell activation does not rely on the presence of MHC class I molecules, as does CAR. Given the success of targeting HERV-K Env as a tumor-associated antigen (TAA), and that nearly all breast cancer cell lines express Kenv protein, the inventors hypothesize that a BiTE specific for Kenv and CD3 (K3Bi) effectively treats metastatic disease as did K-CAR. The inventors have designed and synthesized a K3Bi that has dual specificity for Kenv and CD3. Thus, T cells are directed to target HERV-K+ tumor cells. The inventors have generated, purified, and validated the K3Bi and a CD8 BiTE (K8Bi). This was done using the mAb 6H5 that was also used in the CAR construct (scientific reference 33), and OKT3, an antibody against human CD3 previously used in other BiTEs, which was humanized and connected with a flexible linker plus two C-terminal epitope tags (MYC and FLAG) for purification and staining. A CD8 single chain antibody (scFv) obtained from OKT8 hybridoma cells was generated in the inventors' lab and used to produce K8Bi (VL-VH6H5 linker VH-VLCD8-MYC and FLAG). K3Bi and K8Bi were cloned into the pLJM1-EGFP Lenti or pGEX-6P-1 vector for recombinant protein expression. The capacity of the K3Bi or K8Bi to bind to T cells and HERV-K+ breast cancer cell lines was determined by several immune assays. The inventors found that increased numbers of target cells bound to BiTE with increased BiTE concentrations.

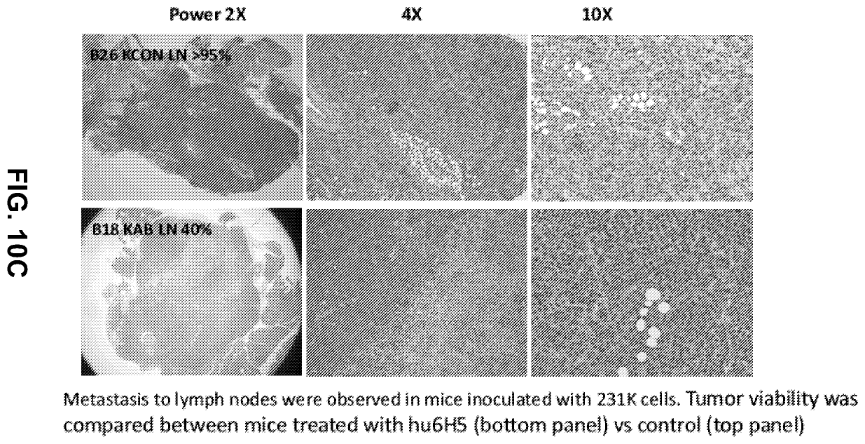

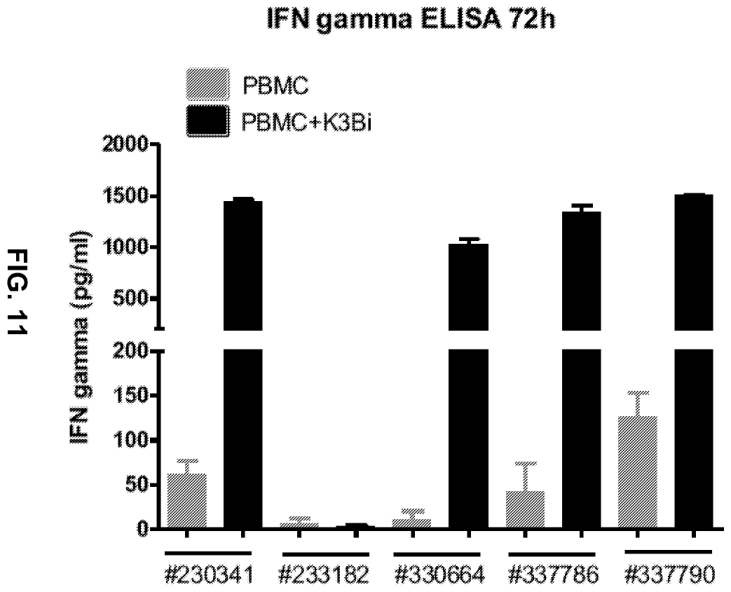

The inventors also examined the capacity of the K3Bi to induce T cell activation, proliferation, production of cytokines, and lysis of target tumor cells. Bulk PBMCs (50,000 per well) from healthy controls co-cultured with K3Bi (0, 1, 10, 100, and 1,000 ng/ml) and tumor cells (5,000 per well) to achieve effector cell: target cell ratios of 10:1 as described in scientific reference 51. One result is shown in FIG. 12. PBMC+MCF-7+K3Bi exhibited increased cancer cell killing compared PBMC+MCF-7 without K3Bi (FIG. 12B). An LDH release assay was used for detection of cell viability and cytotoxicity, as the inventors did previously. See scientific reference 33. Enhanced IFN-γ production, assayed by ELISA (FIG. 12C) was observed in MDA-MB-231, MDA-MB-468, and MCF-7 cells treated with K3Bi. Untreated cells, PBMC only, or BiTE only were used as controls and no IFN-γ production was observed in these control groups.

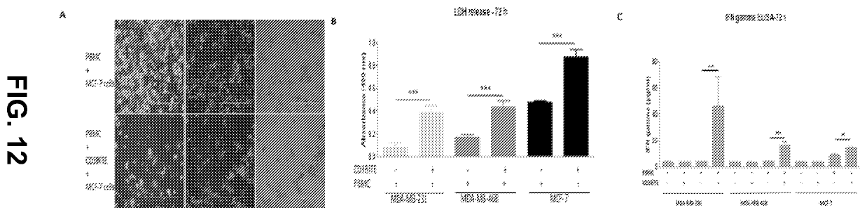

Furthermore, treatment of immunodeficient NSG mice bearing HERV-K positive MDA-MB-231 breast cancer cells with PBMCs and CD3 HERV-K BiTE plus IL-2 or CD8 HERV-K BiTE plus PBMCs plus IL-2 resulted in greatly decreased tumor growth (FIG. 13).

### Example 3

### Staining Results of Normal Donor PBMC's Traduced with CAR-A and CAR-B Lentiviral Vectors

PBMCs from normal donors were transduced with two CAR-T lentiviral vector constructs, K-CAR-A (CAR-A) or K-CAR B (CAR-B). pWPT-GFP with psPAX2 and pMD2g. VH-VLhu6H5-CD8-CD28-4-1BB-CD3zeta. The protocol to generate HERV-Kenv CAR-T cells by an alternate to the Sleeping Beauty transduction process, namely lentiviral transduction, is as follows:
1. Thaw PBMCs (2×107) and deplete monocytes by plastic adherence 1 hour's incubation at 37° ° C. 5% CO2).
2. Culture monocytes depleted PBMCs in RPMI 1640 supplemented with 10% FBS, 100 U/mL penicillin, 100 μg/mL streptomycin (complete medium). Stimulate T cells with anti-CD3/CD28 beads in a 3:1 bead:cell ratio with 40 IU/mL IL-2 for 24 h.
3. Transduce activated T cells with CAR-A or CAR-B lentiviral particles (CD19 CAR as control).
4. Twenty-four hours after transducing, culture T cells in complete medium containing 300 IU/mL IL-2 and γ-irradiated (100 Gy) MDA-MB-231-Kenv (Kenv is the envelope protein of HERV-K) aAPC at a 2:1 aAPC/T cell ratio to stimulate CAR-T cell proliferation. Use γ-irradiated K562-CD19 as the control aAPC.
5. Remove Anti-CD3/CD28 beads on day 5. Replenish CAR-T cells with fresh media containing IL-2 every two-three days.
6. Use CAR-T cells to perform further experiments when proliferation show a decrease from log-phase.

The CAR-A or CAR-B transduced cells were co-cultured with γ-irradiated (100 Gy) MDA MB 231 antigen presenting cells. Soluble IL-2 cytokine (50 U/ml) was added every other day. On day 14 the cells were harvested for staining. They were stained first for 20 minutes at 4° C. with a 1:1000 dilution of BV450 live and dead stain. After 20 min, the cells were washed and stained with K10-AF 488 protein (1 μg/ml), CD4 Amcyan, CD3 Pe cy7, and goat anti human IgG Fc AF 594 antibodies according to manufacturers' recommendations for 30 mins at 4C and washed with PBS. The cells were fixed with 4% PFA for 15-30 mins and washed before analyzing in a flow cytometer. The samples were positive for GFP, as they were transfected with GFP+CAR-A/CAR-B.

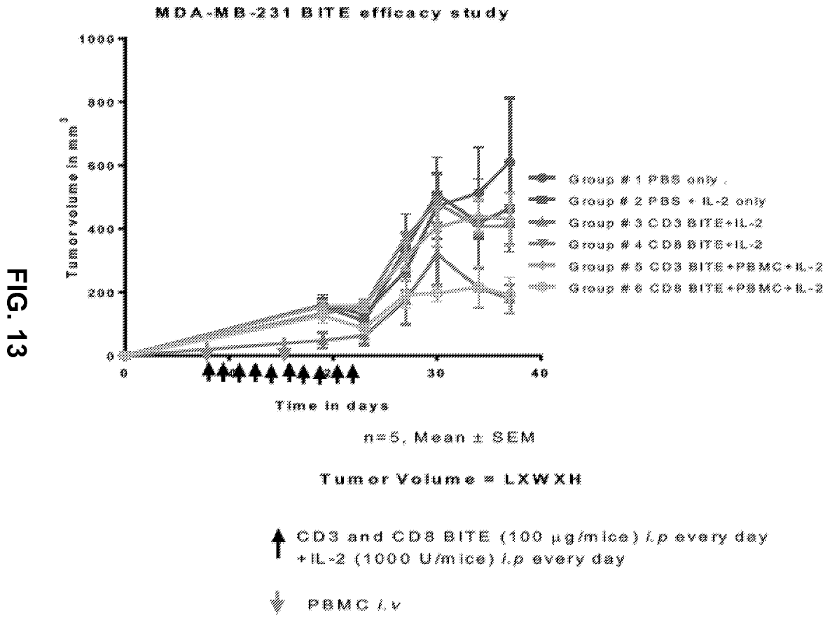

The percentage of CD4+ cells was determined by gating those populations that were negative for BV450 and positive for respective colors. The percentage of CD4−ve (called CD8+ve cells) were gated by selecting those populations that were negative for BV450 and negative for CD4 Amcyan color. The results show that the percentage of CD4+ve PBMC's transduced with CAR-A/CAR-B that get stained with K10 labelled AF488 protein are higher than the percentage of naïve T cells that get stained with K10 labelled AF488 protein (FIG. 14). This shows that T cells transduced with CAR-A or CAR-B are stained with the HERV-K10 protein.

T cells expressing a lentiviral CAR expression vector that bears a humanized or fully human HERV-K scFv will effectively lyse and kill tumor cells from several different cancers. Humanized K-CARs expressed from lentiviral vectors are pan-cancer CAR-Ts.

### Example 4

### A HERV-K Specific Humanized Chimeric Antigen Receptor (K-CAR) Therapy

The inventors have produced a humanized single chain variable fragment (scFv) antibody (Example 1), which was able to bind antigens produced from recombinant HERV-K Env surface fusion protein (KSU) (Example 3 just above) and lysates from MDA-MB-231 breast cancer cells. A CAR produced from this humanized scFv is cloned into a lentiviral vector and is used in combination with therapies that include but are not limited to K-CAR T cells plus checkpoint inhibitors, proinflammatory cytokines such as interleukin (IL)-12 and IL-18, oncolytic viruses, and kinase inhibitors (including but not limited to p-RSK, p-ERK).

### Example 5

### Identification of Human Therapeutic Antibodies (hTAbs) from Very Rare B Cells that Exhibit Strong Target Specificity and High Sensitivity

Generation of fully human therapeutic antibodies from the human adaptive immune system: To directly use B cells from breast cancer patients as a source of high-affinity antibodies, the inventors performed an indirect ELISA or immunoblot with HERV-K Env recombinant fusion protein, which the inventors used to detect anti-HERV-K Env specific responses from several different breast cancer patients. Patients with higher titers of anti-HERV-K antibodies were selected for single B cell experiments. PBMCs from breast cancer patients were polyclonally activated: 1) using irradiated 3T3-CD40L fibroblasts for a period of 2 weeks. This method can efficiently stimulate and expand CD40-B cells to large numbers in high purity (>90%) and induce secretion of their antibodies; and 2) ex vivo with recombinant human IL-21, IL-2, soluble CD40 ligand and anti-APOI for 4 days. This second method can enable secretion from the highest percentage of B cells using minimal culture times. IL-21 is known to promote the differentiation to antibody-secreting cells. See scientific references 53, 54. IL-2 stimulation in vitro can trigger human plasma cell differentiation, which requires appropriate T cell help to reach the induction threshold. See scientific reference 55. sCD40L engages with CD40 expressed on the cell surface of B cells to mimic T cell-mediated activation. See scientific reference 56. Since activation also induces cell death, anti-APOI is used to rescue B cells from Fas-induced apoptosis See scientific reference 57. Few cytotoxic B cells were detected.

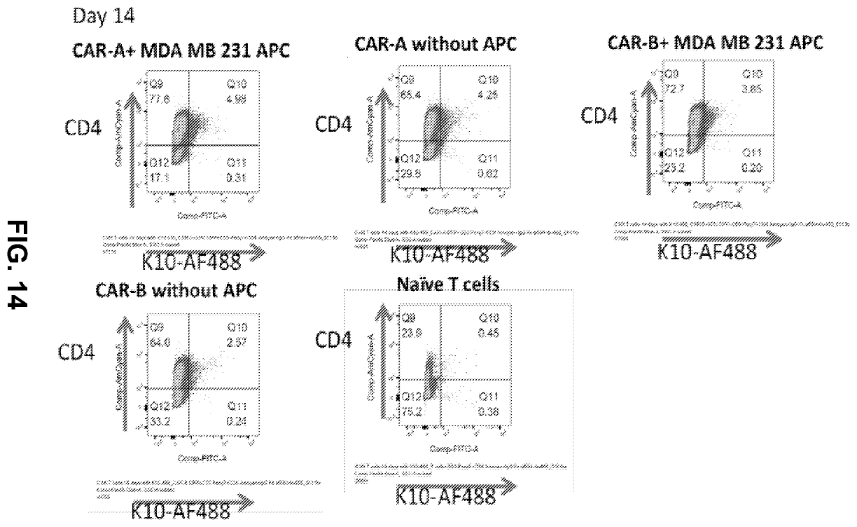

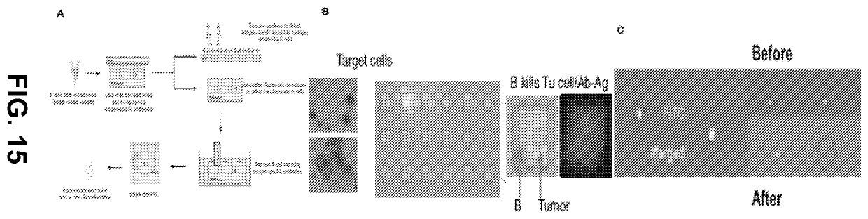

Development of a platform to determine the binding kinetics and cell-to-cell interactions of every cell in a microwell slab. Details of the microengraving process, which enables the screening and monitoring of B cell interactions over time to enable single-cell cloning of antibody-producing B cells, are shown in FIG. 15A. The arrays of nanowells in polydimethyl siloxane (PDMS) are fabricated, and cells from mammospheres from patient breast tumor tissues produced and cultured in the inventors' lab (FIG. 15B; left panel) were used as targets for determining the efficacy of breast cancer cell killing. B cells and mammosphere cells (1:1 ratio) from the same donor were loaded onto a nanowell array (1 cell per well) and the cells allowed to settle via gravity (FIG. 15B middle panel). A dead tumor cell (red color) and B cell are shown in the same well (FIG. 15B). The anti-HERV-K antibody produced by this B cell was detected in the same position of the glass cover slide (right panel, red color square). The single B cell was then picked by a CellCelector (FIG. 15C) for RT-PCR. Our results show that HERV-K specific memory B cells exhibited anti-HERV-K antibody expression as well as cytotoxicity toward their autologous mammosphere cells.

Therapeutic antibody discovery using an in vivo enrichment (IVE) adaptation: Our platform will enable isolation of antibodies that not only bind target cancer cells but can also kill the cells. It will also enable the use of normal donors without memory B cells instead of breast cancer patient donors to generate hTAbs. Since B cells able to produce therapeutic antibodies for treatment are extremely rare even after ex vivo enrichment, the inventors developed the following platform to identify very rare hTAbs:

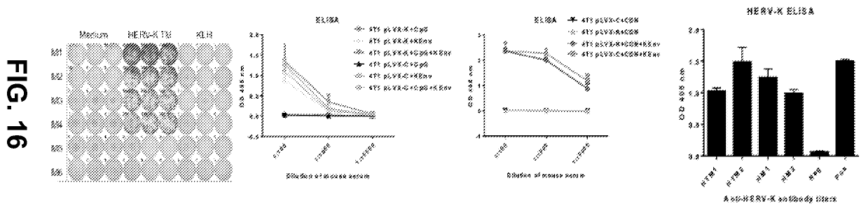

Groups (N=10/group) of wild type Balb/c mice (female, 6-week-old) are immunized on day 1 and boosted on week 3 and week 5. ELISPOT are used to determine IFN-y secretion by CD8+ T cells obtained from immunized mice (FIG. 16A). ELISA assays (FIGS. 16B, 16C, and 16D) are used to detect the titers of anti-HERV-K IgG in immunized mouse sera.

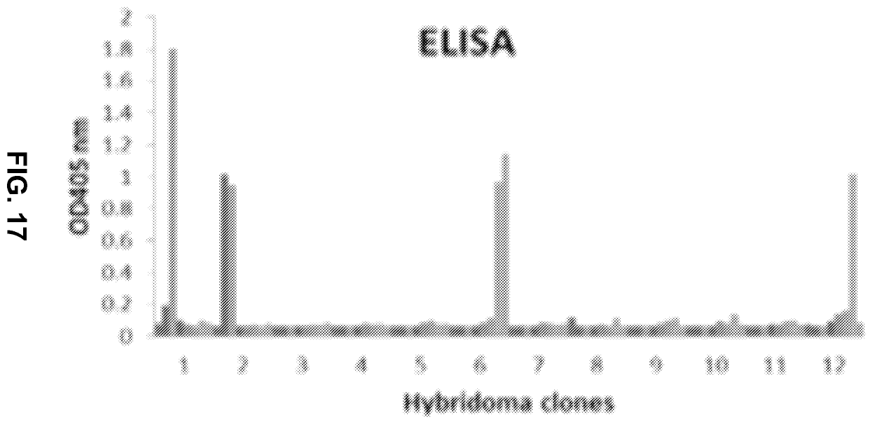

Example 5.1. Adapt an in vivo enrichment technique (IVE: ≈20-fold enhancement) in SCID/beige mice, allowing for rapid expansion and B cell activation, with a goal of producing large numbers of antigen-specific plasmablasts. See FIG. 11A. This platform will produce fully human antibodies from B cells in as short a time as 8 days. As a proof of principle, the inventors developed an IVE technique to produce fully human anti-Zika antibodies in hybridoma cells generated from splenocytes on day 8 fusion with MFP-2 partner cells (FIG. 17A and FIG. 17B).

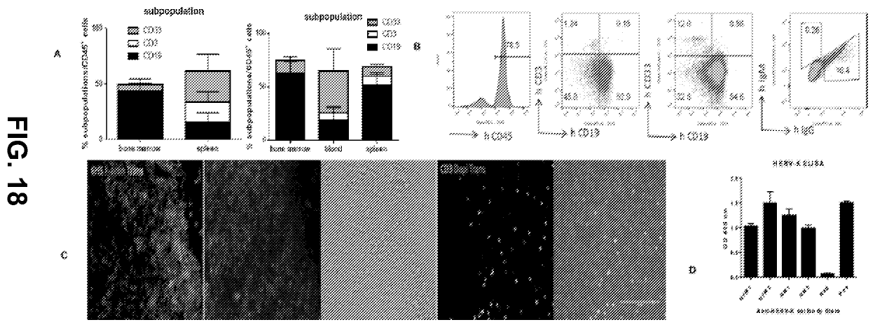

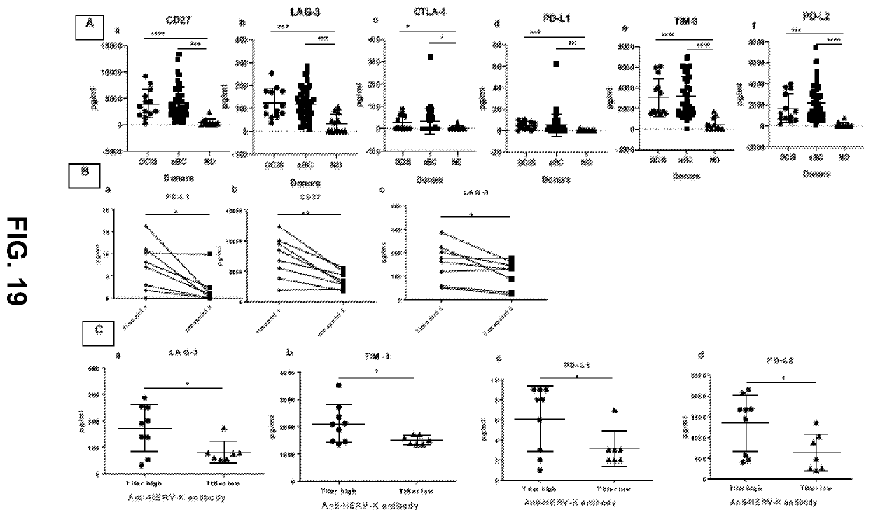

Recently, humanized mice (HM) and human tumor mice (HTM) were successfully generated by intravenous injection of CD34+ cells (1-2×10⁵/mouse) for HM generation and immunization with HERV-K SU or PD-L1 recombined fusion proteins. The inventors also co-implanted CD34+ hematopoietic stem cells with 5×10⁴-3×10⁶ breast cancer cells triple negative breast cancer patient derived xenografts (TNBC PDX cells, or MDA-MB-231 or MDA-MB-468 TNBC cells) in the mammary fat pad for HTM generation. The percentage of hCD19 or hCD45 cells is higher in mice after a longer period of post-inoculation with CD34 cells (FIG. 18A and FIG. 18B). Exposure to antigen was associated with HERV-K expression in the tumor, and a higher antibody titer was detected (HTM 2: 40 days vs. HTM 1: 30 days; FIG. 18C and FIG. 18D). Importantly, this indicates that HTMs can produce anti-HERV-K antibodies in mice inoculated with breast cancer cells. This finding prompted us to explore the use of HM or/and HTM to generate fully hTAbs, and especially to use normal donors who were never exposed to antigen. NSG mice, which lack T-, B-, and NK cell activity, are considered as ideal candidates to establish HM. Mice with a higher engraftment rate of human CD45+ cells than was seen in earlier studies (FIG. 18B), without any significant toxicity, were developed recently.

Protocol 1. For donors who have cancer with a higher titer of antibodies, the inventors use the protocol as in FIG. 17A using HM instead SCID/beige mice. PBMCs (50×10⁶) from breast cancer patients are polyclonally activated by IL-21, IL-2, soluble CD40 ligand and anti-APO1, and premixed with antigens (HERV-K or PD-L1; 100 μg). B cells isolated from the above PBMCs using an EasySep™ Human B Cell Enrichment Kit (Stemcell Technologies) by negative selection are co-injected with CD34 cells in the mice treated with busulfan. See scientific reference 61. (Fisher: 30 mg/kg intraperitoneally) on day 0. Mice are treated with cytokine cocktails (days 1, 4, and 7) and boosted by antigens on day 2. This protocol can be completed relatively quickly (8 days).

Protocol 2. For normal donors who do not have cancer and who have no memory B cells, the inventors use Protocol 1 with modifications: Mice are treated with cytokine cocktails (days 1, 7, and 14) and boosted by antigens on day 14 and day 21. Sera are collected from mice and binding affinity is tested by ELISA. After increased antibody titers are detected, spleens are harvested, analyzed, and used to make hybridomas. Higher antibody titers were detected in mice using IVE Protocol 2 on week 2.

Example 5.2. After IVE, half of the spleen is harvested and used for flow cytometric analysis, microengraving and other analyses. Flow cytometric analysis of B cell surface and intracellular markers and CFSE labeling (Invitrogen CellTrace CFSE kit) is performed using the following: Anti-CD19 PECy5, anti-CD27 allophycocyanin, anti-CD38 PECy7, anti-IgG FITC, or anti-IgM PE isotype controls of mouse IgG1k conjugated to FITC, PE, PECy5, PECy7, Alexa 700, or allophycocyanin (all from BD Bioscience). Negative magnetic immunoaffinity bead separation (Miltenyi Biotec) is used to isolate total CD19+ B cells from spleen and stimulate with CpG2006 (10 ng/ml; Oligos, Inc.) in the presence of recombinant human B cell activating factor (BAFF; 75 ng/ml; GenScript), IL-2 (20 IU/ml), IL-10 (50 ng/ml), and IL-15 (10 ng/ml) (all from BD Biosciences) for 72 hours. Tumor-killing B cells directly from Protocol 1 or 2 are determined using our multi-well microengraving platform (up to 400,000 wells: FIG. 15), with their autologous tumor cells or HERV-K+TNBC cells as target cells. Cells that not only produce antibodies but are also able to bind antigen and kill cancer cells are determined as in FIG. 15.

Example 5.3. The inventors then develop human hybridoma cells to ensure long-term antibody availability. To develop a fully human hybridoma, MFP-2 cells are used as a partner to generate hybridomas with the remaining half of the spleen using ClonaCell™M-HY (Stemcell Technologies Inc.,) following their protocol. Polyethylene glycol (PEG) is used for fusing human lymphocytes with MFP-2 cells and a methylcellulose-based semi-solid media in this kit is used for cloning and selection of hybridoma cells. The clones that grow out after selection are pipetted into 96 well plates and screened for reactivity to HERV-K Env protein by ELISA. The positive clones' isotypes are determined using a Human IgG Antibody Isotyping Kit from Thermo Fisher Scientific. The clones are then adapted to serum-free media conditions and expanded. Hybridoma supernatant is harvested, and antibody is purified using Hi-Trap protein A or protein G columns, depending on the isotype of the human antibody. Protein A columns are known to have high affinity to antibodies of the isotype-IgG1, 2, and 4, and variable binding to antibodies of the isotype IgM, whereas Protein G columns are known to exhibit high binding to antibodies of the isotype-IgG1, 2, 3 and 4, but do not bind IgM antibodies.

Example 5.4. The inventors evaluate the antitumor efficacy of candidate B cells obtained from the above protocols in vitro, including effects on cell growth, proliferation, and apoptosis, as the inventors do routinely in our lab. In vivo studies to evaluate the efficacy of the hTAbs in immunodeficient mouse models are also done to evaluate efficacy, using breast cancer cell lines and primary tumor cells, and compared with matched uninvolved control breast cells.

### Example 6

### Combination Therapy

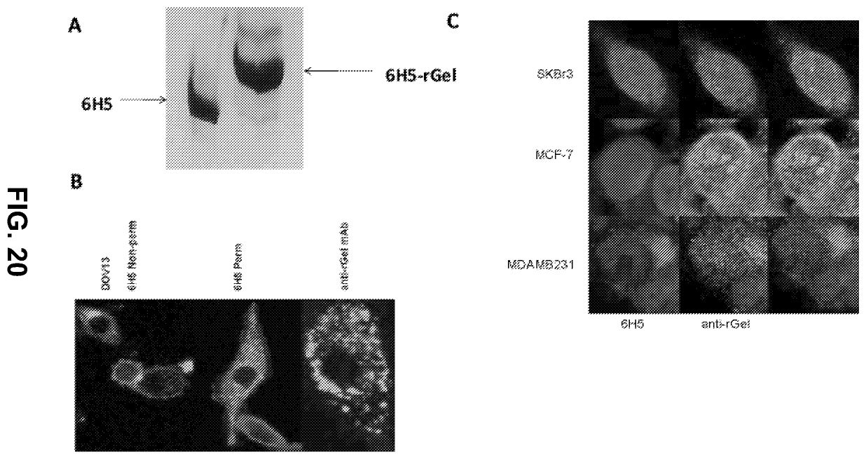

The inventors' breast cancer data from strongly support the potential for combination therapy approaches involving HERV-K. Humanized and fully human antibodies targeting HERV-K will therefore enhance checkpoint blockade antibody treatment efficacy. Effective combined cancer therapies include but are not limited to combinations of (a) HERV-K hTAb (1.5 mg/kg), (b) K-CAR, (c) K-BiTE, (d) HERV-K shRNAs or CRISPR/Cas9 genome editing technology to knock down HERV-K gene expression, (e) or preventative or therapeutic HERV-K vaccines, including full-length and truncated HERV-K Env proteins and HERV-K Env peptides, and (a) anti-ICP antibody (FIG. 19), (b) cancer chemotherapy, (c) 5-azacytidine, 5-aza-2′-deoxycytidine, or other epigenetic modulating agents, such as DNA methyltransferase inhibitors (DNMTi) and histone deacetylase inhibitors (HDACi), (d) EMT inhibitors, (e) inhibitors of cell migration or invasion, (f) induction of S or G2 phase cell cycle arrest, (g) inhibitors of PI3K/AKT/mTOR or MAPK/ERK signaling pathways, or (h) signal transduction to HIF1α.

### Example 7

### Sequence Data anti-CD3 and CD8 BiTE

| OKT8 Heavy chains: (Anti-CD8 mAb sequence). | | |
|---|---|---|
| >H1 | | |
| ttt gag gtc cag ctg cag cag tct ggg gca gag ctt gtg aag cca ggg gcc tca gtc aag | | |
| F E V Q L Q Q S G A E L V K P G A S V K | | |
| ttg tcc tgc aca gct tct ggc ttc aac att aaa gac acc tat ata cac ttc gtg agg cag | | |
| L S C T A S G F N I K D T Y I H F V R Q | | |
| agg cct gaa cag ggc ctg gag tgg att gga agg att gat cct gcg aat gat aat act tta | | |
| R P E Q G L E W I G R I D P A N D N T L | | |
| tat gcc tca aag ttc cag ggc aag gcc act ata aca gca gac aca tca tcc aac aca gcc | | |
| Y A S K F Q G K A T I T A D T S S N T A | | |
| tac atg cac ctc tgc agc ctg aca tct ggg gac act gcc gtc tat tac tgt ggt aga ggt | | |
| Y M H L C S L T S G D T A V Y Y C G R G | | |
| tat ggt tac tac gta ttt gac cac ttg ggc caa ggc nnt nnn nnt nnc ann ntn nnn nnn (SEQ ID NO: 21) | | |
| Y G Y Y V F D H L G Q G X X X X X X X X (SEQ ID NO: 22) | | |
| >H3 | | |
| tnt cag gtg cag ctg aag cag tct ggg gca gag ctt gtg aag cca ggg gcc tca gtc aag | | |
| X Q C Q L K Q S G A E L V K P G A S V K | | |
| ttg tcc tgc aca gct tct ggc ttc aac att aaa gac acc tat ata cac ttc gtg agg cag | | |
| L S C T A S G F N I K D T Y I H F V R Q | | |
| agg cct gaa cag ggc ctg gag tgg att gga agg att gat cct gcg aat gat aat act tta | | |
| R P E Q G L E W I G R I D P A N D N T L | | |
| tat gcc tca aag ttc cag ggc aag gcc act ata aca gca gac aca tca tcc aac aca gcc | | |
| Y A S K F Q G K A T I T A D T S S N T A | | |
| tac atg cac ctc tgc agc ctg aca tct ggg gac act gcc gtc tat tac tgt ggt aga ggt | | |
| Y M H L C S L T S G D T A V Y Y C G R G | | |
| tat ggt tac tac gta ttt gac cac tgg ggc caa ggc acc act ctc aca ntn ncn nnn (SEQ ID NO: 23) | | |
| Y G Y Y V F D H W G Q G T T L T X X X (SEQ ID NO: 24) | | |
| >H6 | | |
| ttt gag gtc cag ctg cag cag tct ggg gca gag ctt gtg aag cca ggg gcc tca gtc aag | | |
| F E V Q L Q Q S G A E L V K P G A S V K | | |
| ttg tcc tgc aca gct tct ggc ttc aac att aaa gac acc tat ata cac ttc gtg agg cag | | |
| L S C T A S G F N I K D T Y I H F V R Q | | |
| agg cct gaa cag ggc ctg gag tgg att gga agg att gat cct gcg aat gat aat act tta | | |
| R P E Q G L E W I G R I D P A N D N T L | | |
| tat gcc tca aag ttc cag ggc aag gcc act ata aca gca gac aca tca tcc aac aca gcc | | |
| Y A S K F Q G K A T I T A D T S S N T A | | |
| tac atg cac ctc tgc agc ctg aca tct ggg gac act gcc gtc tat tac tgt ggt aga ggt | | |
| Y M H L C S L T S G D T A V Y Y C G R G | | |
| tat ggt tac tac gta ttt gac cac tgg ggc caa ggc acc act ctc aca ntn ncn nna (SEQ ID NO: 25) | | |
| Y G Y Y V F D H W G Q G T T L T X X X (SEQ ID NO: 26) | | |
| >H7 | | |
| tcag gtc caa ctg cag cag cct ggg gca gag ctt gtg aag cca ggg gcc tca gtc aag ttg | | |
| Q V Q L Q Q P G A E L V K P G A S V R L | | |
| tcc tgc aca gct tct ggc ttc aac att aaa gac acc tat ata cac ttc gtg agg cag agg | | |
| S C T A S G F N I K D T Y I H F V R Q R | | |
| cct gaa cag ggc ctg gag tgg att gga agg att gat cct gcg aat gat aat act tta tat | | |
| P E Q G L E W I G R I D P A N D N T L Y | | |
| gcc tca aag ttc cag ggc aag gcc act ata aca gca gac aca tca tcc aac aca gcc tac | | |
| A S K F Q G K A T I T A D T S S N T A Y | | |
| atg cac ctc tgc agc ctg aca tct ggg gac act gcc gtc tat tac tgt ggt aga ggt tat | | |
| M H L C S L T S G D T A V Y Y C G R G Y | | |
| ggt tac tac gta ttt gac cac tgg ggc caa ggc acc act ctc aca tnt nnn (SEQ ID NO: 27) | | |
| G Y Y V F D H W G Q G T T L T X X (SEQ ID NO: 28) | | |
| Alignment | | |
| H3 | TNTCAGGTGCAGCTGAAGCAGTCTGGGGCAGAGCTTGTGAAGCCAGGGGCCTCAGTCAAG | 60 |
| H6 | TTTGAGGTCCAGCTGCAGCAGTCTGGGGCAGAGCTTGTGAAGCCAGGGGCCTCAGTCAAG | 60 |
| H7 | --TCAGGTCCAACTGCAGCAGCCTGGGGCAGAGCTTGTGAAGCCAGGGGCCTCAGTCAAG | 58 |
| H1 | TTTGAGGTCCAGCTGCAGCAGTCTGGGGCAGAGCTTGTGAAGCCAGGGGCCTCAGTCAAG | 60 |
| | ** *** **.***.***** ************************************** | |
| H3 | TTGTCCTGCACAGCTTCTGGCTTCAACATTAAAGACACCTATATACACTTCGTGAGGCAG | 120 |
| H6 | TTGTCCTGCACAGCTTCTGGCTTCAACATTAAAGACACCTATATACACTTCGTGAGGCAG | 120 |
| H7 | TTGTCCTGCACAGCTTCTGGCTTCAACATTAAAGACACCTATATACACTTCGTGAGGCAG | 118 |
| H1 | TTGTCCTGCACAGCTTCTGGCTTCAACATTAAAGACACCTATATACACTTCGTGAGGCAG | 120 |
| | ************************************************************ | |
| H3 | AGGCCTGAACAGGGCCTGGAGTGGATTGGAAGGATTGATCCTGCGAATGATAATACTTTA | 180 |
| H6 | AGGCCTGAACAGGGCCTGGAGTGGATTGGAAGGATTGATCCTGCGAATGATAATACTTTA | 180 |
| H7 | AGGCCTGAACAGGGCCTGGAGTGGATTGGAAGGATTGATCCTGCGAATGATAATACTTTA | 178 |
| H1 | AGGCCTGAACAGGGCCTGGAGTGGATTGGAAGGATTGATCCTGCGAATGATAATACTTTA | 180 |
| | ************************************************************ | |
| H3 | TATGCCTCAAAGTTCCAGGGCAAGGCCACTATAACAGCAGACACATCATCCAACACAGCC | 240 |
| H6 | TATGCCTCAAAGTTCCAGGGCAAGGCCACTATAACAGCAGACACATCATCCAACACAGCC | 240 |
| H7 | TATGCCTCAAAGTTCCAGGGCAAGGCCACTATAACAGCAGACACATCATCCAACACAGCC | 238 |
| H1 | TATGCCTCAAAGTTCCAGGGCAAGGCCACTATAACAGCAGACACATCATCCAACACAGCC | 240 |
| | ************************************************************ | |
| H3 | TACATGCACCTCTGCAGCCTGACATCTGGGGACACTGCCGTCTATTACTGTGGTAGAGGT | 300 |
| H6 | TACATGCACCTCTGCAGCCTGACATCTGGGGACACTGCCGTCTATTACTGTGGTAGAGGT | 300 |
| H7 | TACATGCACCTCTGCAGCCTGACATCTGGGGACACTGCCGTCTATTACTGTGGTAGAGGT | 298 |
| H1 | TACATGCACCTCTGCAGCCTGACATCTGGGGACACTGCCGTCTATTACTGTGGTAGAGGT | 300 |
| | ************************************************************ | |
| H3 | TATGGTTACTACGTATTTGAC-CACTGGGGCCAAGGCACCAC---TCTCACANTNNCNNN | 356 |
| H6 | TATGGTTACTACGTATTTGAC-CACTGGGGCCAAGGCACCAC---TCTCACANTNNCNNN | 356 |
| H7 | TATGGTTACTACGTATTTGAC-CACTGGGGCCAAGGCACCAC---TCTCACATNTNNNTN | 354 |
| H1 | TATGGTTACTACGTATTTGACTCACTTGGGCCAAGGCNNTNNNNNTNNCANNNTNNNNNN | 360 |
| | ********************* **** ********** * .** ...* *.* | |
| H3 | N- | 357 (SEQ ID NO: 24) |
| H6 | A- | 357 (SEQ ID NO: 26) |
| H7 | -- | --- (SEQ ID NO: 28) |
| H1 | NN | 362 (SEQ ID NO: 22) |
| OKT8 Kappa chains: | | |
| >K4 | | |
| tttt gac att gtg ctg acc caa tct cct gct tcc tta gct gta tct ctg ggg cag agg gcc | | |
| F D I V L T Q S P A S L A V S L G Q R A | | |
| acc atc tca tac agg gcc agc aaa agt gtc agt aca tct ggc tat agt tat atg cac tgg | | |
| T I S Y R A S K S V S T S G Y S Y M H W | | |
| aac caa cag aaa cca gga cag cca ccc aga ctc ctc atc tat ctt gta tcc aac cta gaa | | |
| N Q Q K P G Q P P R L L I Y L V S N L E | | |
| tct ggg gtc cct gcc agg ttc agt ggc agt ggg tct ggg aca gac ttc acc ctc aac atc | | |
| S G V P A R F S G S G S G T D F T L N I | | |
| cat cct gtg gag gag gag gat gct gca acc tat tac tgt cag cac att agg gag ctt aca | | |
| H P V E E E D A A T Y Y C Q H I R E L T | | |
| cgt tcg gag ggg gac caa gcn nnn nnn (SEQ ID NO: 29) | | |
| R S E G D Q X X X (SEQ ID NO: 30) | | |
| >K6 | | |
| tntt gat att gtg cta act cag tct cct gct tcc tta gct gta tct ctg ggg cag agg gcc | | |
| X D I V L T Q S P A S L A V S L G Q R A | | |
| acc atc tca tac agg gcc agc aaa agt gtc agt aca tct ggc tat agt tat atg cac tgg | | |
| T I S Y R A S K S V S T S G Y S Y M H W | | |
| aac caa cag aaa cca gga cag cca ccc aga ctc ctc atc tat ctt gta tcc aac cta gaa | | |
| N Q Q K P G Q P P R L L I Y L V S N L E | | |
| tct ggg gtc cct gcc agg ttc agt ggc agt ggg tct ggg aca gac ttc acc ctc aac atc | | |
| S G V P A R F S G S G S G T D F T L N I | | |
| cat cct gtg gag gag gag gat gct gca acc tat tac tgt cag cac att agg gag ctt aca | | |
| H P V E E E D A A T Y Y C Q H I R E L T | | |
| cgn nga (SEQ ID NO: 31) | | |
| X X (SEQ ID NO: 32) | | |
| >K8 | | |
| tgac atc cag ctg act cag tct cct gct tcc tta gct gta tct ctg ggg cag agg gcc acc | | |
| D I Q L T Q S P A S L A V S L G Q R A T | | |
| atc tca tac agg gcc agc aaa agt gtc agt aca tct ggc tat agt tat atg cac tgg aac | | |
| I S Y R A S K S V S T S G Y S Y M H W N | | |
| caa cag aaa cca gga cag cca ccc aga ctc ctc atc tat ctt gta tcc aac cta gaa tct | | |
| Q Q K P G Q P P R L L I Y L V S N L E S | | |
| ggg gtc cct gcc agg ttc agt ggc agt ggg tct ggg aca gac ttc acc ctc aac atc cat | | |
| G V P A R F S G S G S G T D F T L N I H | | |
| cct gtg gag gag gag gat gct gct gac cta tta gct gtc agc aca tta tgg gag ctt aca | | |
| P V E E E D A A D L L A V S T L W E L T | | |
| cgt tcg gnn nnn (SEQ ID NO: 33) | | |
| R S X X (SEQ ID NO: 34) | | |
| Alignment | | |
| K4 | TTTTGACATTGTGCTGACCCAATCTCCTGCTTCCTTAGCTGTATCTCTGGGGCAGAGGGC | 60 |
| K6 | TNTTGATATTGTGCTAACTCAGTCTCCTGCTTCCTTAGCTGTATCTCTGGGGCAGAGGGC | 60 |
| K8 | ---TGACATCCAGCTGACTCAGTCTCCTGCTTCCTTAGCTGTATCTCTGGGGCAGAGGGC | 57 |
| | *** ** :***.** **.************************************** | |
| K4 | CACCATCTCATACAGGGCCAGCAAAAGTGTCAGTACATCTGGCTATAGTTATATGCACTG | 120 |
| K6 | CACCATCTCATACAGGGCCAGCAAAAGTGTCAGTACATCTGGCTATAGTTATATGCACTG | 120 |
| K8 | CACCATCTCATACAGGGCCAGCAAAAGTGTCAGTACATCTGGCTATAGTTATATGCACTG | 117 |
| | ************************************************************ | |
| K4 | GAACCAACAGAAACCAGGACAGCCACCCAGACTCCTCATCTATCTTGTATCCAACCTAGA | 180 |
| K6 | GAACCAACAGAAACCAGGACAGCCACCCAGACTCCTCATCTATCTTGTATCCAACCTAGA | 180 |
| K8 | GAACCAACAGAAACCAGGACAGCCACCCAGACTCCTCATCTATCTTGTATCCAACCTAGA | 177 |
| | ************************************************************ | |
| K4 | ATCTGGGGTCCCTGCCAGGTTCAGTGGCAGTGGGTCTGGGACAGACTTCACCCTCAACAT | 240 |
| K6 | ATCTGGGGTCCCTGCCAGGTTCAGTGGCAGTGGGTCTGGGACAGACTTCACCCTCAACAT | 240 |
| K8 | ATCTGGGGTCCCTGCCAGGTTCAGTGGCAGTGGGTCTGGGACAGACTTCACCCTCAACAT | 237 |
| | ************************************************************ | |
| K4 | CCATCCTGTGGAGGAGGAGGATGCTGCA-ACCTATTA-CTGTCAGCACATTA-GGGAGCT | 297 |
| K6 | CCATCCTGTGGAGGAGGAGGATGCTGCA-ACCTATTA-CTGTCAGCACATTA-GGGAGCT | 297 |
| K8 | CCATCCTGTGGAGGAGGAGGATGCTGCTGACCTATTAGCTGTCAGCACATTATGGGAGCT | 297 |
| | ***************************: ******** ************** ******* | |
| K4 | TACACGTTCGGAGGGGGACCAAGCNNNNNNN--- | 328 (SEQ ID NO: 30) |
| K6 | TACACGNNGANAANNNNNNNNNNCNNNNTNNNNN | 331 (SEQ ID NO: 32) |
| K8 | TACACGTTCGGNNNNNTNNCTNNINCNNNNNNNC | 331 (SEQ ID NO: 34) |

### Order of the IgG Domains

VL-VH6H5---VH-VLhuCD3 or CD8+c-myc tag+FLAG or VL-VHhu6H5---VH-VLhuCD3 or huCD8+c-myc tag+FLAG

```
CD8 BiTE:
acc ggt atg gat atc gag ctg acc cag agc cct agc agc ctg gcc gtg tca ctg ggc cag
  T G M D I E L T Q S P S S L A V S L G Q
aga gcc acc atc agc tgc aga gcc tcc gag agc gtg gat agc cac ggc acc agc ctg atg
  R A T I S C R A S E S V D S H G T S L M
cac tgg tat cag cag aag ccc ggc cag ccc ccc aag ttc ctg atc tac cgg gcc agc aac
  H W Y Q Q K P G Q P P K F L I Y R A S N
ctg gaa agc ggc atc ccc gcc aga ttt tcc ggc agc ggc agc aga acc gac ttc acc ctg
  L E S G I P A R F S G S G S R T D F T L
acc atc aac ccc gtg gag aca gac gac gtg gcc atc tac tac tgc cag cag agc aac gag
  T I N P V E T D D V A I Y Y C Q Q S N E
gac cct ccc acc ttt ggc gga ggc acc aag ctg gaa ctg aag gag ggc gga gga agc gga
  D P P T F G G G T K L E L K E G G G S G
ggg gga gga tct ggc gga ggc ggc agc gcc cag gtg aag ctg cag cag agc ggc cct gat
  G G G S G G G G S A Q V K L Q Q S G P D
ctg gtg aag cct ggc gcc agc gtg aag atc agc tgc aag gcc agc ggc tac agc ttc acc
  L V K P G A S V K I S C K A S G Y S F T
ggc tac tac atg cac tgg gtg aaa cag agc cac ggc aag agc ctg gaa tgg atc ggc aga
  G Y Y M H W V K Q S H G K S L E W I G R
gtg aac ccc aat agc ggc ggc acc agc tac aac cag aag ttc aag gac aag gcc atc ctg
  V N P N S G G T S Y N Q K F K D K A I L
acc gtg gac aag agc agc agv acc gcc tac atg gaa ctg cgg agc ctg acc agc gag gac
  T V D K S S S T A Y M E L R S L T S E D
agc gcc gtg tac tac tgv gcc cgg tcv aag ggv aac tac ttc tac gcc atg gac tac tgg
  S A V Y Y C A R S K G N Y F Y A M D Y W
ggc cag ggc acc acc gtg acc gtg tct agc agc ggc ggc ggc gga agc gaa gtg cag ctg
  G Q G T T V T V S S S G G G G S E V Q L
cag cag tcc gga gca gaa ctg gtg aaa ccc gga gcc agt gtg aag ctg agc tgt aca gcc
  Q Q S G A E L V K P G A S V K L S C T A
tcc ggc ttt aat atc aag gac acc tac atc cac ttc gtg cgc caa cgg cca gaa cag ggt
  S G F N I K D T Y I H F V R Q R P E Q G
ctg gag tgg att ggc agg atc gat cca gca aat gat aac acc ctg tac gca agc aaa ttt
  L E W I G R I D P A N D N T L Y A S K F
cag ggc aaa gcc acg ata acc gcc gat aca tct agt aat acg gct tac atg cac ctc tgc
  Q G K A T I T A D T S S N T A Y M H L C
tcc ctg act tcc ggg gac acc gcc gtg tat tat tgc ggg cgc gga tac ggt tac tac gtg
  S L T S G D T A V Y Y C G R G Y G Y Y V
ttc gat cat tgg ggt cag ggc acc acc ctc aca gtc tcc agt gca gga ggc gga ggt agt
  F D H W G Q G T T L T V S S A G G G G S
gga ggc gga ggc tct ggc ggt ggc ggg tca gac ata gtg ctg aca cag tct ccc gcc agt
  G G G G S G G G G S D I V L T Q S P A S
ctg gca gtg agc ctt ggc cag agg gct aca att tcc tat agg gcg tct aaa tcc gtt agc
  L A V S L G Q R A T I S Y R A S K S V S
act tca ggt tac tct tat atg cac tgg aac cag cag aag cct ggt cag ccc ccc agg ctt
  T S G Y S Y M H W N Q Q K P G Q P P R L
ctt att tac ctg gtc agc aat ctc gag tct ggc gtg ccc gct aga ttt tcc ggc agc ggg
  L I Y L V S N L E S G V P A R F S G S G
agt ggg act gac ttc act ctg aac atc cac cca gtg gag gaa gag gat gcc gca acc tac
  S G T D F T L N I H P V E E E D A A T Y
tat tgt cag cat atc cgg gaa ttg acc aga tca gag ggc gga cct tcc tgg aag gag cag
  Y C Q H I R E L T R S E G G P S W K E Q
aag ctg att agc gag gaa gat ctg gat tat aag gac gac gat gac aaa tga gaa ttc (SEQ ID NO: 35)
  K L I S E E D L D Y K D D D D K - E F (SEQ ID NO: 36)
CD3 BiTE:
acc ggt atg gat atc gag ctg acc cag agc cct agc agc ctg gcc gtg tca ctg ggc cag_
  T G M D I E L T Q S P S S L A V S L G Q
aga gcc acc atc agc tgc aga gcc tcc gag agc gtg gat agc cac ggc acc agc ctg atg
  R A T I S C R A S E S V D S H G T S L M
cac tgg tat cag cag aag ccc ggc cag ccc ccc aag ttc ctg atc tac cgg gcc agc aac
  H W Y Q Q K P G Q P P K F L I Y R A S N
ctg gaa agc ggc atc ccc gcc aga ttt tcc ggc agc ggc agc aga acc gac ttc acc ctg
  L E S G I P A R F S G S G S R T D F T L
acc atc aac ccc gtg gag aca gac gac gtg gcc atc tac tac tgc cag cag agc aac gag
  T I N P V E T D D V A I Y Y C Q Q S N E
gac cct ccc acc ttt ggc gga ggc acc aag ctg gaa ctg aag gag ggc gga gga agc gga
  D P P T F G G G T K L E L K E G G G S G
ggg gga gga tct ggc gga ggc ggc agc gcc cag gtg aag ctg cag cag agc ggc cct gat
  G G G S G G G G S A Q V K L Q Q S G P D
ctg gtg aag cct ggc gcc agc gtg aag atc agc tgc aag gcc agc ggc tac agc ttc acc
  L V K P G A S V K I S C K A S G Y S F T
ggc tac tac atg cac tgg gtg aaa cag agc cac ggc aag agc ctg gaa tgg atc ggc aga
  G Y Y M H W V K Q S H G K S L E W I G R
gtg aac ccc aat agc ggc ggc acc agc tac aac cag aag ttc aag gac aag gcc atc ctg
  V N P N S G G T S Y N Q K F K D K A I L
acc gtg gac aag agc agc agc acc gcc tac atg gaa ctg cgg agc ctg acc agc gag gac
  T V D K S S S T A Y M E L R S L T S E D
agc gcc gtg tac tac tgc gcc cgg tcc aag ggc aac tac ttc tac gcc atg gac tac tgg
  S A V Y Y C A R S K G N Y F Y A M D Y W
ggc cag ggc acc acc gtg acc gtg tct agc agc ggc ggc ggc gga agc gag gtt cag ctg
  G Q G T T V T V S S S G G G G S E V Q L
gtg gag tct ggc ggt ggc ctg gtg cag cca ggg ggc tca ctc cgt ttg tcc tgt gca gct
  V E S G G G L V Q P G G S L R L S C A A
tct ggc tac tcc ttt acc ggc tac act atg aac tgg gtg cgt cag gcc cca ggt aag ggc
  S G Y S F T G Y T M N W V R Q A P G K G
ctg gaa tgg gtt gca ctg att aat cct tat aaa ggt gtt tcc acc tat aac cag aaa ttc
  L E W V A L I N P Y K G V S T Y N Q K F
aag gat cgt ttc acg ata tcc gta gat aaa tcc aaa aac aca gcc tac ctg caa atg aac
  K D R F T I S V D K S K N T A Y L Q M N
agc ctg cgt gct gag gac act gcc gtc tat tat tgt gct aga agc gga tac tac ggc gat
  S L R A E D T A V Y Y C A R S G T T G D
agc gac tgg tat ttt gac gtc tgg ggt caa gga acc ctg gtc acc gtc tcc tcg ggt gga
  S D W Y F D V W G Q G T L V T V S S G G
ggc ggt tca ggc gga ggt ggc tct ggc ggt ggc gga tcg gat atc cag atg acc cag tcc
  G G S G G G G S G G G G S D I Q M T Q S
ccg agc tcc ctg tcc gcc tct gtg ggc gat agg gtc acc atc acc tgt cgt gcc agt cag
  P S S L S A S V G D R V T I T C R A S Q
gac atc cgt aat tat ctc aac tgg tat caa cag aaa cca gga aaa gct ccg aaa cta ctg
  D I R N Y L N W Y Q Q K P G K A P K L L
att tac tat acc tcc cgc ctg gag tct gga gtc cct tct cgc ttc tct ggt tct ggt tct
  I Y Y T S R L E S G V P S R F S G S G S
ggg acg gat tac act ctg acc atc agc agt ctg caa ccg gag gac ttc gca act tat tac
  G T D Y T L T I S S L Q P E D F A T Y Y
tgt cag caa ggt aat act ctg ccg tgg acg ttc gga cag ggc acc aag gtg gag atc aaa
  C Q Q G N T L P W T F G Q G T K V E I K
gag cag aag ctg att agc gag gaa gat ctg gat tat aag gac gac gat gac aaa tga gaa
  E Q K L I S E E D L D Y K D D D D K - E
ttc (SEQ ID NO: 37)
F (SEQ ID NO: 38)
```

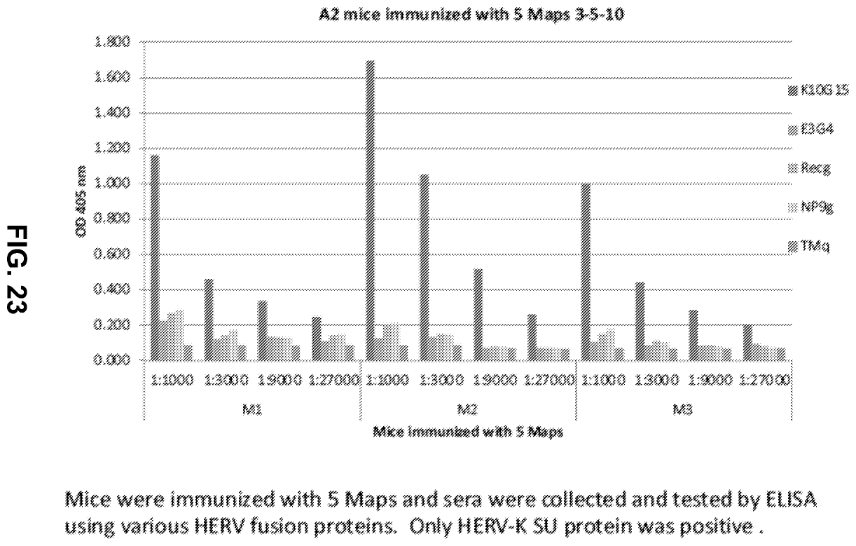

Mice were immunized with 5 Maps and sera were collected and tested by ELISA using various HERV fusion proteins (FIG. 23). Only HERV-K SU protein was positive. Hybridoma cells were generated from the mice immunized with 5 Maps and a scFv was selected having the sequence below.

| scFv against MAPs of HERV-K (sequence for anti-HERV-K mAb). | | |
|---|---|---|
| 1 | ATGGCCCAGGTGCAACTGCAGCAGTCAGGGGCTGAGCTTGTGAAGCCTGGGGCTTCAGTG | 60 |
| | M A Q V Q L Q Q S G A E L V K P G A S V | |
| 61 | AAGATGTCCTGCAAGGCTTCTGGCTACACCTTCACCAGCTACTGGATAACCTGGGTGAAG | 120 |
| | K M S C K A S G Y T E T S Y W I T W V K | |
| 121 | CAGAGGCCTGGACAAGGCCTTGAGTGGATTGGAGATATTTATCCTGGTAGTGGTAGTACT | 180 |
| | Q R P G Q G L E W I G D I Y P G S G S T | |
| 181 | AACTACAATGAGAAGTTCAAGAGCAAGGCCACACTGACTGTAGACACATCCTCCAGCACA | 240 |
| | N Y N E K F K S K A T L T V D T S S S T | |
| 241 | GCCTACATGCAGCTCAGCAGCCTGACATCTGAGGACTCTGCGGTCTATTACTGTGCAAGA | 300 |
| | A Y M Q L S S L T S E D S A V Y Y C A R | |
| 301 | TGGCGGGACGGGTACTATGCTATGGACTACTGGGGCCAAGGGACCACGGTCACCGTCTCC | 360 |
| | W R D G Y Y A M D Y W G Q G T T V T V S | |
| 361 | TCAAGTGAAGGCGGTTCAGGCGGAGGTTGCTCTGGCGGTGGCGGATCGGACATCGAGCTC | 420 |
| | S S E G G S G G G C S G G G G S D I E L | |
| 421 | ACTCAGTCTCCAACCACCATGGCTGCATCTCCCGGGGAGAAGATCACTATCACCTGCAGT | 480 |
| | T Q S P T T M A A S P G E K I T I T C S | |
| 481 | GCCAGCTCAAGTATAAGTTCCAATTACTTGCATTGGTATCAGCAGAAGCCAGGATTCTCC | 540 |
| | A S S S I S S N Y L H W Y Q Q K P G F S | |
| 541 | CCTAAACTCTTGATTTATAGGACATCCAATCTGGCTTCTGGAGTCCCAGCTCGCTTCAGT | 600 |
| | P K L L I Y R T S N L A S G V P A R F S | |
| 601 | GGCAGTGGGTCTGGGACCTCTTACTCTCTCACAATTGGCACCATGGAAGCTGAAGATGTT | 660 |
| | G S G S G T S Y S L T I G T M E A E D V | |
| 661 | GCCACTTACTACTGCCAGCAGGGTAGTAGTATACCATTCACGTTCGGCTCGGGGACGAAG | 720 |
| | A T Y Y C Q Q G S S I P F T F G S G T K | |
| 721 | TTGGAGCTGAAACGGGCGGGCCGCAGGTGCGCCGGTGCCGTATCCG | 766 (SEQ ID NO: 45) |
| | L E L K R A G R R C A G A V S | (SEQ ID NO: 46) |

### Example 8

### Humanized Antibodies Targeting HERV-K that Can be Used for ADCs to Deliver the Drugs into Cancer Cells and Tumors

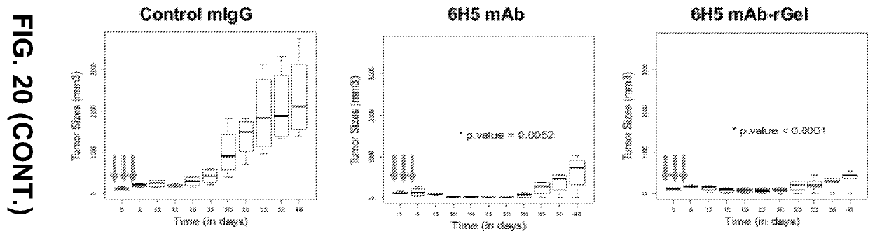

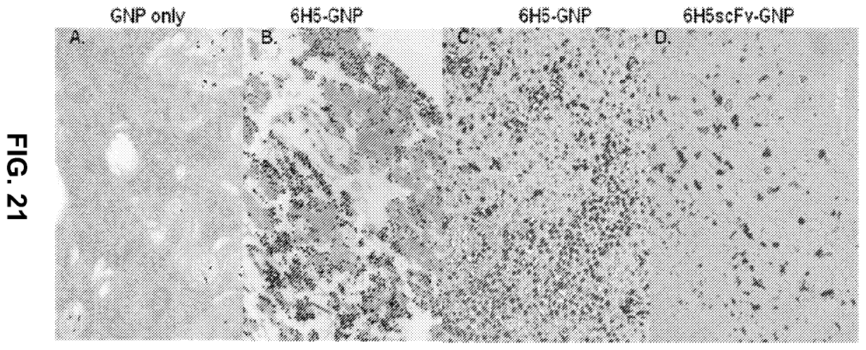

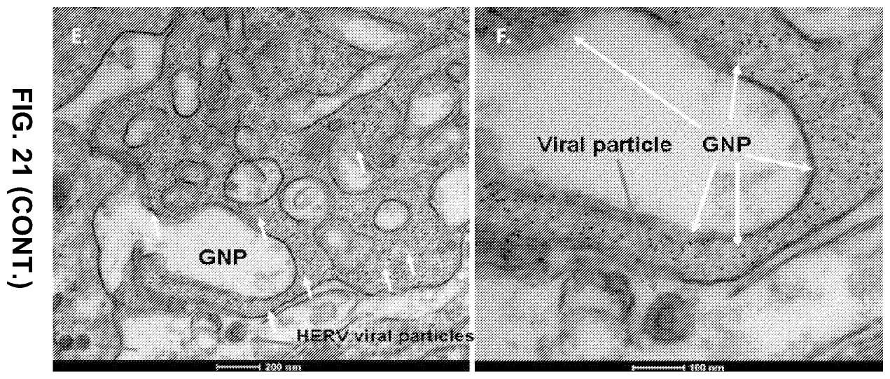

Recombinant gelonin (r-Gel) toxin was conjugated with 6H5 (FIG. 20A). r-Gel was detected in OVCAR3 (FIG. 20B), SKBr3, MCF-7, and MDA-MB-231 cells (FIG. 20C) after 1 hour's internalization using anti-r-Gel antibody. Furthermore, gold nanoparticles (GNPs) were detected after 2 hours incubation with naked GNP (FIG. 21A) or 6H5-GNP (FIG. 21B) by transmission electron microscopy (TEM) in MDA-MB-231 cells. GNPs were detected in MDA-MB-231 (FIGS. 21C, 21E, and 21F) or SKBr3 (FIG. 21D) of tumors isolated from mice 24 hours post-intravenous-injection with the 6H5-GNP (FIGS. 21C, 21E, and 21F) or 6H5scFV-GNP (FIG. 21D) using a silver enhancement assay. GNPs generate heat that kills targeted tumor cells when they are placed in a radiofrequency field.

### Example 9

### In Vivo Imaging of Anti-HERV-K Antibodies in Tumor Nodules of Mice

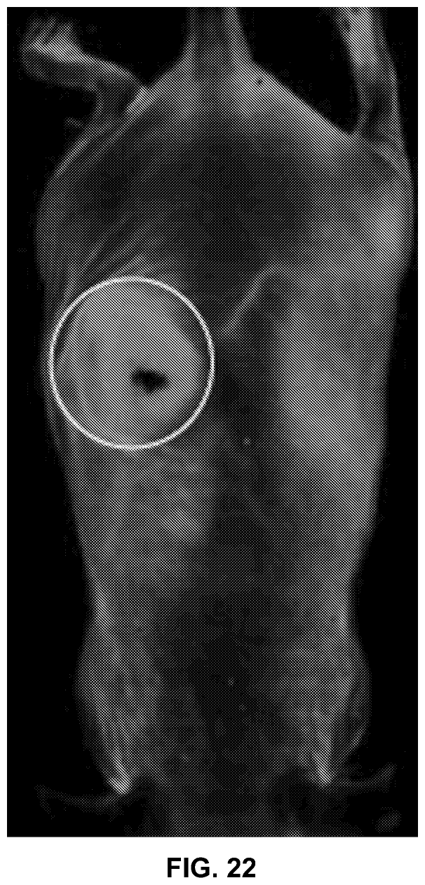

A higher density of 6H5 was detected in tumor nodules from mice 24 hours post-intravenous-injection with the anti-HERV-K-Alexa647 conjugate 6H5-Alexa647 (red color) by in vivo imaging using a Nuance system (FIG. 22).

### List of Embodiments

Specific compositions and methods of HERV-K antibody therapeutics. The scope of the invention should be defined solely by the claims. A person having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

Among the embodiments provided in this specification are the following:
1. An isolated antibody that binds to human endogenous retrovirus-K (HERV-K), comprising a heavy chain variable region (HCVR) and a light chain variable region (LCVR). Humanized anti-HERV-K antibody is able reduce tumor growth, especially reduce metastasis to lung, lymph nodes and other organs.
2. The antibody according to embodiment 1, comprising a humanized or human framework region.
3. The antibody according to embodiment 1, wherein the antibody is an HERV-K antagonist.
4. An isolated nucleic acid comprising a nucleotide sequence encoding the HCVR, the LCVR, or a combination thereof of embodiment 1.
5. An expression vector comprising the nucleic acid of embodiment 4.
6. A host cell transformed with an expression vector of embodiment 5.
7. A method of producing an antibody comprising a HCVR, a LCVR, or a combination thereof, the method comprising: growing the host cell of embodiment 1, under conditions such that the host cell expresses the antibody comprising the HCVR, the LCVR, or a combination thereof; and isolating the antibody comprising the HCVR, the LCVR, or combination thereof.
10. A method of treating cancer in a mammal, comprising administering an effective amount of the antibody according to embodiment 1 to a mammal in need thereof.
11. A method for treating cancer comprising administering, to an individual in need thereof, an effective amount of an ADC comprising an antibody comprising a heavy chain variable region (VH) and a light chain variable region (VL), wherein: the VH region comprises a CDR1, a CDR2, and a CDR3, and the VL region comprises a CDR1, a CDR2, and a CDR3, wherein the antibody is conjugated to a cytotoxic drug, an auristatin or a functional peptide analog or derivate thereof via a linker.
12. The method of embodiment 11, wherein the ADC is administered in combination with one or more additional therapeutic agents.
13. The method of embodiment 11, wherein the one or more additional therapeutic agents includes a chemotherapeutic agent.
14. The method of embodiment 11, wherein the cancer is selected from the group consisting of melanoma, chronic lymphocytic leukemia, breast cancer, pancreatic cancer, head and neck cancer, ovarian cancer, cervical cancer, colorectal cancer, testicular cancer, stomach cancer, kidney cancer, endometrial cancer, uterine cancer, bladder cancer, prostate cancer, esophageal cancer, liver cancer, and non-small cell lung cancer.
The humanized antibody developed for CAR T, CAR NK, and BiTE studies.
15. The method of embodiment 11, wherein the antibody is a full-length antibody.
16. The method of embodiment 11, wherein the antibody is a human monoclonal IgG1 or IgG4 antibody.
17. The method of embodiment 11, wherein the auristatin is monomethyl auristatin E (MMAE).
18. The method of embodiment 11, wherein the auristatin is monomethyl auristatin F (MMAF).
19. The method of embodiment 11, wherein the cytotoxic drug is emtansine (DM1).
20. The method of embodiment 11, wherein the cytotoxic drug is ozagamicin (calicheamicin).
21. The method of embodiment 11, wherein the cytotoxic drug is deruxtecan (DXd).
22. The method of embodiment 11, wherein the cytotoxic drug is govitecan (SN-38).
23. The method of embodiment 11, wherein the cytotoxic drug is mafodotin (MMAF).
24. The method of embodiment 11, wherein the cytotoxic drug is duocarmazine (duocarmycin).
25. The method of embodiment 11, wherein the cytotoxic drug is BAT8001 (maytansinoid) soravtansine (DM4).
26. The method of embodiment 11, wherein the cytotoxic drug is tesirine (PBD).
27. The method of embodiment 11, wherein the linker is attached to sulphydryl residues of the antibody obtained by partial reduction of the antibody.
28. The method of embodiment 11, wherein the linker-auristatin is vcMMAF or vcMMAE.
29. The early detection, metastasis, or HERV-K plus immune checkpoint biomarkers, substantially as described herein.
30. Antibody-based therapeutics, substantially as described herein,
32. Cancer cells overexpressing HERV-K as targets for the anti-HERV-K humanized antibodies and ADCs of the invention.
33. The hu6H5 clones (FWJ1 and FWJ2) generated from bacteria (HUM1 and HUM2) or mammalian cells.
34. A BiTE directed against T cell CD3 or CD8 and a humanized scFv against the tumor-associated antigen HERV-K, comprising antibodies targeting either CD3 or CD8 and HERV-K.
35. T cells expressing a lentiviral CAR expression vector that bears a humanized or fully human HERV-K scFv.
36. A humanized single chain variable fragment (scFv) antibody able to bind antigens produced from recombinant HERV-K Env surface fusion protein (KSU) and lysates from cancer cells expressed HERV-K Env proteins.
37. A CAR produced from the humanized scFv of embodiment 28.
38. A CAR produced from the humanized scFv of embodiment 28, which is cloned into a lentiviral vector.
39. A CAR produced from the humanized scFv of embodiment 28, which is cloned into a lentiviral vector, for use in combination therapies.
44. An improved in vivo enrichment method for rapid expansion and B cell activation for donors who have no memory B cells, comprising the steps of: treating mice with cytokine cocktails on days 1, 7, and 14, and boosting the mice by antigens on days 14 and 21.
45. Cells that not only produce antibodies, but the antibodies are also able to bind antigen and kill cancer cells, and cells that express the antigen are able to be killed by the antibodies.
47. A significantly enhanced expression of six circulating immune checkpoint proteins in the plasma of breast cancer patients.
50. A method of blockading of the immunosuppressive domain (ISD) with immune checkpoint inhibitors of HERV-K.
51. The method of embodiment 42, wherein the immune checkpoint inhibitors of HERV-K are selected from the group consisting of monoclonal antibodies and drugs targeting the ISD of HERV-K.
52. Humanized and fully human antibodies targeting HERV-K, for use in enhancing checkpoint blockade antibody treatment efficacy.
53. A method to produce new antibodies from mice immunized with 5 multiple antigen peptides (MAPS) that are generated from HERV-K SU protein produced by cancer patients.
54. A method to produce to produce HERV-K CAR A: VH-VLhu6H5-CD8-CD28-4-1BB-CD3zeta.

### REFERENCES

A person having ordinary skill in the molecular biological art of can use the following patents, patent applications, and scientific references as guidance to predictable results when making and using the invention:

### Patent References

U.S. Pat. No. 9,243,055 (Wang-Johanning). This patent discloses and claims cancer diagnostics and therapy. Methods and compositions for detecting, preventing, and treating HERV-K+ cancers are provided. One method is for preventing or inhibiting cancer cell proliferation by administering to a subject a cancer cell proliferation blocking or reducing amount of a HERV-K env protein binding antibody.
International Pat. Publ. WO 2014/186469 (Board of Regents, the University of Texas System). This patent publication concerns methods and compositions for immunotherapy using a modified T cell comprising a chimeric antigen receptor (CAR). CAR-expressing T-cells are produced using electroporation in conjunction with a transposon-based integration system to produce a population of CAR-expressing cells that require minimal ex vivo expansion or that can be directly administered to patients for cancer treatment.

### Scientific References

1. Lander E S, Linton L M, Birren B, et al. Initial sequencing and analysis of the human genome. Nature. 2001; 409: 860-921. HERVs are well-known as genomic repeat sequences, with many copies in the genome, such that approximately 8% of the human genome is of retroviral origin.
2. Hughes J F, Coffin J M. Evidence for genomic rearrangements mediated by human endogenous retroviruses during primate evolution. Nat Genet. 2001; 29: 487-489. HERVs originated from thousands of ancient integration events which incorporated retrovirus DNA into germline cells.
3. Larsson E, Kato N, Cohen M. Human endogenous proviruses. Curr Top Microbiol Immunol. 1989; 148: 115-132. The most biologically active HERVs are members of the HERV-K family. HERV-K has a complete sequence capable of expressing all the elements needed for a replication-competent retrovirus
4. Ono M, Yasunaga T, Miyata T, Ushikubo H. Nucleotide sequence of human endogenous retrovirus genome related to the mouse mammary tumor virus genome. J Virol. 1986; 60: 589-598. The most biologically active HERVs are members of the HERV-K family. HERV-K has a complete sequence capable of expressing all the elements needed for a replication-competent retrovirus
5. Kraus B, Fischer K, Buchner S M, et al. Vaccination directed against the human endogenous retrovirus-K envelope protein inhibits tumor growth in a murine model system. PLOS One. 2013; 8: e72756.
6. Denne M, Sauter M, Armbruester V. Licht J D, Roemer K, Mueller-Lantzsch N. Physical and functional interactions of human endogenous retrovirus proteins Np9 and rec with the promyelocytic leukemia zinc finger protein. J Virol. 2007; 81: 5607-5616.
7. Fischer S, Echeverria N, Moratorio G, et al. Human endogenous retrovirus np9 gene is over expressed in chronic lymphocytic leukemia patients. Leuk Res Rep. 2014; 3: 70-72.
8. Li M R R, Yin B. Li J, Chivukula R, Lin K, Lu Y, Shen J, Chang D Z, Li D, Johanning G L, Wang-Johanning F. Down-regulation of human endogenous retrovirus type K (HERV-K) viral env RNA in pancreatic cancer cells decreases cell proliferation and tumor growth. Clinical Cancer Research. 2017; In press.
9. Feng Wang-Johanning I L, Gary Johanning, Ming Li, Raghu Chivukula, Guoqing Wu. Tumor microenvironment predicts aggressive breast cancer: Combination of HERV-K, immune checkpoint and activation status of CD8+ T cells. Cancer Res. 2017; 77: Abstract nr LB-221.
10. Zhou F, Li M, Wei Y, et al. Activation of HERV-K Env protein is essential for tumorigenesis and metastasis of breast cancer cells. Oncotarget. 2016; 7: 84093-84117.
11. Wang-Johanning F, Radvanyi L, Rycaj K, et al. Human endogenous retrovirus K triggers an antigen-specific immune response in breast cancer patients. Cancer Res. 2008; 68: 5869-5877. The inventors observed the expression of HERVs, especially HERV-K sequences, in breast, lung, prostate, ovarian, colon, pancreatic, and other solid tumors.
12. Zhao J, Rycaj K, Geng S, et al. Expression of Human Endogenous Retrovirus Type K Envelope Protein is a Novel Candidate Prognostic Marker for Human Breast Cancer. Genes Cancer. 2011; 2: 914-922. The inventors observed the expression of HERVs, especially HERV-K sequences, in breast, lung, prostate, ovarian, colon, pancreatic, and other solid tumors.
13. Wang-Johanning F, Liu J, Rycaj K, et al. Expression of multiple human endogenous retrovirus surface envelope proteins in ovarian cancer. Int J Cancer. 2007; 120: 81-90.
14. Wang-Johanning F, Li M, Esteva F J, et al. Human endogenous retrovirus type K antibodies and mRNA as serum biomarkers of early-stage breast cancer. Int J Cancer. 2014; 134: 587-595.
15. Wang-Johanning F, Frost A R, Johanning G L, et al. Expression of human endogenous retrovirus k envelope transcripts in human breast cancer. Clin Cancer Res. 2001; 7: 1553-1560.
16. Wang-Johanning F, Frost A R, Jian B, Epp L, Lu D W, Johanning G L. Quantitation of HERV-K env gene expression and splicing in human breast cancer. Oncogene. 2003; 22: 1528-1535. The inventors observed the expression of HERVs, especially HERV-K sequences, in breast, lung, prostate, ovarian, colon, pancreatic, and other solid tumors.
17. Wang-Johanning F, Frost A R, Jian B, et al. Detecting the expression of human endogenous retrovirus E envelope transcripts in human prostate adenocarcinoma. Cancer. 2003; 98: 187-197. The inventors observed the expression of HERVs, especially HERV-K sequences, in breast, lung, prostate, ovarian, colon, pancreatic, and other solid tumors.
18. Wallace T A, Downey R F, Seufert C J, et al. Elevated HERV-K mRNA expression in PBMC is associated with a prostate cancer diagnosis particularly in older men and smokers. Carcinogenesis. 2014; 35: 2074-2083.
19. Downey R F, Sullivan F J, Wang-Johanning F. Ambs S, Giles F J, Glynn S A. Human endogenous retrovirus K and cancer: Innocent bystander or tumorigenic accomplice? Int J Cancer. 2015; 137: 1249-1257.
20. Johanning G L, Malouf G G, Zheng X, et al. Expression of human endogenous retrovirus-K is strongly associated with the basal-like breast cancer phenotype. Sci Rep. 2017; 7: 41960. The inventors observed the expression of HERVs, especially HERV-K sequences, in breast, lung, prostate, ovarian, colon, pancreatic, and other solid tumors. They also found that the expression of HERV-K env transcripts in breast cancer was specifically associated with basal breast cancer, a particularly aggressive subtype.
21. Serafino A, Balestrieri E, Pierimarchi P, et al. The activation of human endogenous retrovirus K (HERV-K) is implicated in melanoma cell malignant transformation. Exp Cell Res. 2009; 315: 849-862.
22. Oricchio E, Sciamanna I, Beraldi R, Tolstonog G V, Schumann G G, Spadafora C. Distinct roles for LINE-1 and HERV-K retroelements in cell proliferation, differentiation and tumor progression. Oncogene. 2007; 26: 4226-4233.
23. Gonzalez-Cao M, Iduma P, Karachaliou N, Santarpia M, Blanco J, Rosell R. Human endogenous retroviruses and cancer. Cancer Biol Med. 2016; 13: 483-488.
24. Kleiman A, Senyuta N, Tryakin A, et al. HERV-K(HML-2) GAG/ENV antibodies as indicator for therapy effect in patients with germ cell tumors. Int J Cancer. 2004; 110: 459-461.
25. Buscher K, Trefzer U, Hofmann M, Sterry W, Kurth R, Denner J. Expression of human endogenous retrovirus K in melanomas and melanoma cell lines. Cancer Res. 2005; 65: 4172-4180.
26. Seifarth W, Skladny H, Krieg-Schneider F, Reichert A, Hehlmann R, Leib-Mosch C. Retrovirus-like particles released from the human breast cancer cell line T47-D display type B- and C-related endogenous retroviral sequences. J Virol. 1995; 69: 6408-6416.
27. Etkind P R, Lumb K, Du J, Racevskis J. Type 1 HERV-K genome is spliced into subgenomic transcripts in the human breast tumor cell line T47D. Virology. 1997; 234: 304-308.
28. Ono M, Kawakami M, Ushikubo H. Stimulation of expression of the human endogenous retrovirus genome by female steroid hormones in human breast cancer cell line T47D. J Virol. 1987; 61: 2059-2062.
29. Ejthadi H D, Martin J H, Junying J, et al. A novel multiplex RT-PCR system detects human endogenous retrovirus-K in breast cancer. Arch Virol. 2005; 150: 177-184.
30. Wang-Johanning F, Rycaj K, Plummer J B, et al. Immunotherapeutic potential of anti-human endogenous retrovirus-K envelope protein antibodies in targeting breast tumors. J Natl Cancer Inst. 2012; 104: 189-210. The inventors showed that the HERV-K Env protein is commonly expressed on the surface of breast cancer cells. The inventors observed the expression of HERVs, especially HERV-K sequences, in breast, lung, prostate, ovarian, colon, pancreatic, and other solid tumors 30-34.
31. Li M, Radvanyi L, Yin B. et al. Downregulation of Human Endogenous Retrovirus Type K (HERV-K) Viral env RNA in Pancreatic Cancer Cells Decreases Cell Proliferation and Tumor Growth. Clin Cancer Res. 2017; 23: 5892-5911. The inventors observed the expression of HERVs, especially HERV-K sequences, in breast, lung, prostate, ovarian, colon, pancreatic, and other solid tumors.
32. Rycaj K, Plummer J B, Yin B, et al. Cytotoxicity of human endogenous retrovirus K-specific T cells toward autologous ovarian cancer cells. Clin Cancer Res. 2015; 21: 471-483. The inventors observed the expression of HERVs, especially HERV-K sequences, in breast, lung, prostate, ovarian, colon, pancreatic, and other solid tumors.
33. Zhou F, Krishnamurthy J, Wei Y, et al. Chimeric antigen receptor T cells targeting HERV-K inhibit breast cancer and its metastasis through downregulation of Ras. Oncoimmunology. 2015; 4: e1047582. The inventors observed the expression of HERVs, especially HERV-K sequences, in breast, lung, prostate, ovarian, colon, pancreatic, and other solid tumors.
34. Zhou F, Li M, Wei Y, et al. Activation of HERV-K Env protein is essential for tumorigenesis and metastasis of breast cancer cells. Oncotarget. 2016.
35. Krishnamurthy J, Rabinovich B A, Mi T, et al. Genetic Engineering of T Cells to Target HERV-K, an Ancient Retrovirus on Melanoma. Clin Cancer Res. 2015; 21: 3241-3251. The inventors observed the expression of HERVs, especially HERV-K sequences, in breast, lung, prostate, ovarian, colon, pancreatic, and other solid tumors.
36. Chae Y K, Chang S. Ko T, et al. Epithelial-mesenchymal transition (EMT) signature is inversely associated with T-cell infiltration in non-small cell lung cancer (NSCLC). Sci Rep. 2018; 8: 2918.
37. Lemaitre C, Tsang J, Bireau C, Heidmann T, Dewannieux M. A human endogenous retrovirus-derived gene that can contribute to oncogenesis by activating the ERK pathway and inducing migration and invasion. PLOS Pathog. 2017; 13: e1006451.
38. Li M, Radvanyi LG, Yin B, et al. Down-regulation of human endogenous retrovirus type K (HERV-K) viral env RNA in pancreatic cancer cells decreases cell proliferation and tumor growth. Clin Cancer Res. 2017.
39. Helsen C W, Hammill J A, Lau V W C, et al. The chimeric TAC receptor co-opts the T cell receptor yielding robust anti-tumor activity without toxicity. Nat Commun. 2018; 9: 3049.
40. Burstein H J, Winer E P. Refining therapy for human epidermal growth factor receptor 2-positive breast cancer: T stands for trastuzumab, tumor size, and treatment strategy. J Clin Oncol. 2009; 27: 5671-5673.
41. Natsume A, Niwa R, Satoh M. Improving effector functions of antibodies for cancer treatment: Enhancing ADCC and CDC. Drug Des Devel Ther. 2009; 3: 7-16.
42. Spector N L, Blackwell K L. Understanding the mechanisms behind trastuzumab therapy for human epidermal growth factor receptor 2-positive breast cancer. J Clin Oncol. 2009; 27: 5838-5847.
43. Feldhaus M J, Siegel R W, Opresko L K, et al. Flow-cytometric isolation of human antibodies from a nonimmune Saccharomyces cerevisiae surface display library. Nat Biotechnol. 2003; 21: 163-170.
44. Mazor Y, Van Blarcom T, Mabry R, Iverson B L. Georgiou G. Isolation of engineered, full-length antibodies from libraries expressed in Escherichia coli. Nat Biotechnol. 2007; 25: 563-565.
45. Zhou Y, Zou H, Zhang S, Marks J D. Internalizing cancer antibodies from phage libraries selected on tumor cells and yeast-displayed tumor antigens. J Mol Biol. 2010; 404: 88-99.
46. Lanzavecchia A, Corti D, Sallusto F. Human monoclonal antibodies by immortalization of memory B cells. Curr Opin Biotechnol. 2007; 18: 523-528.
47. Smith K, Garman L, Wrammert J, et al. Rapid generation of fully human monoclonal antibodies specific to a vaccinating antigen. Nat Protoc. 2009; 4: 372-384.
48. Scheid J F, Mouquet H, Feldhahn N, et al. Broad diversity of neutralizing antibodies isolated from memory B cells in HIV-infected individuals. Nature. 2009; 458: 636-640.
49. Meijer P J, Andersen P S, Haahr Hansen M, et al. Isolation of human antibody repertoires with preservation of the natural heavy and light chain pairing. J Mol Biol. 2006; 358: 764-772.
50. Kontermann R E, Brinkmann U. Bispecific antibodies. Drug Discov Today. 2015.
51. Zhang P, Shi B, Gao H, et al. An EpCAM/CD3 bispecific antibody efficiently eliminates hepatocellular carcinoma cells with limited galectin-1 expression. Cancer Immunol Immunother. 2014; 63: 121-132.
52. Seitz S, Schneider C K, Malotka J, et al. Reconstitution of paired T cell receptor alpha- and beta-chains from microdissected single cells of human inflammatory tissues. Proc Natl Acad Sci U S A. 2006; 103: 12057-12062.
53. Ettinger R, Sims G P, Fairhurst A M, et al. IL-21 induces differentiation of human naive and memory B cells into antibody-secreting plasma cells. J Immunol. 2005; 175: 7867-7879.
54. Good K L, Bryant V L, Tangye S G. Kinetics of human B cell behavior and amplification of proliferative responses following stimulation with IL-21. J Immunol. 2006; 177: 5236-5247.
55. Hipp N. Symington H, Pastoret C, et al. IL-2 imprints human naive B cell fate towards plasma cell through ERK/ELK1-mediated BACH2 repression. Nat Commun. 2017; 8: 1443.
56. Elgueta R, Benson M J, de Vries V C, Wasiuk A, Guo Y, Noelle R J. Molecular mechanism and function of CD40/CD40L engagement in the immune system. Immunol Rev. 2009; 229: 152-172.
57. Koncz G, Hueber A O. The Fas/CD95 Receptor Regulates the Death of Autoreactive B Cells and the Selection of Antigen-Specific B Cells. Front Immunol. 2012; 3: 207.
59. Song Y C, Liu S J. A TLR9 agonist enhances the anti-tumor immunity of peptide and lipopeptide vaccines via different mechanisms. Sci Rep. 2015; 5: 12578.
60. Chiappinelli K B, Strissel P L, Desrichard A, et al. Inhibiting DNA Methylation Causes an Interferon Response in Cancer via dsRNA Including Endogenous Retroviruses. Cell. 2015; 162: 974-986.
61. Choi B, Chun E, Kim M, et al. Human B cell development and antibody production in humanized NOD/SCID/IL-2Rgamma(null) (NSG) mice conditioned by busulfan. J Clin Immunol. 2011; 31: 253-264.
62. Morozov V A, Dao Thi V L, Denner J. The transmembrane protein of the human endogenous retrovirus-K (HERV-K) modulates cytokine release and gene expression. PLOS One. 2013; 8: e70399.
63. Liu H, Moynihan K D, Zheng Y, et al. Structure-based programming of lymph-node targeting in molecular vaccines. Nature. 2014; 507: 519-522.
64. Kontsekova E, Zilka N, Kovacech B, Novak P. Novak M. First-in-man tau vaccine targeting structural determinants essential for pathological tau-tau interaction reduces tau oligomerisation and neurofibrillary degeneration in an Alzheimer's disease model. Alzheimers Res Ther. 2014; 6: 44.
65. Novak P, Schmidt R, Kontsekova E, et al. Safety and immunogenicity of the tau vaccine AADvac1 in patients with Alzheimer's disease: a randomised, double-blind, placebo-controlled, phase 1 trial. Lancet Neurol. 2017; 16: 123-134.
66. Robinson-McCarthy L R, McCarthy K R, Raaben M, et al. Reconstruction of the cell entry pathway of an extinct virus. PLOS Pathog. 2018; 14: e1007123.

1-28. (canceled)

29. An HERV-K-binding protein comprising an antibody heavy chain variable domain and an antibody light chain variable domain comprising
SEQ ID NOs: 6 and 9, or
SEQ ID NOs: 7 and 10,
respectively.

30. The HERV-K-binding protein of claim 29, wherein the HERV-K-binding protein comprises an antibody.

31. The HERV-K-binding protein of claim 30, wherein the antibody is of human IgG isotype subtype.

32. The HERV-K-binding protein of claim 31, wherein the antibody comprises a heavy chain and a light chain comprising SEQ ID NOs: 42 and 44, respectively.

33. The HERV-K-binding protein of claim 30, wherein the antibody is conjugated to a cytotoxic drug.

34. The HERV-K-binding protein of claim 29, wherein the HERV-K-binding protein comprises an scFv.

35. The HERV-K-binding protein of claim 34, wherein the scFv comprises SEQ ID NO:12 or 13.

36. The HERV-K-binding protein of claim 29, wherein the HERV-K-binding protein is a chimeric antigen receptor (CAR).

37. The HERV-K-binding protein of claim 29, wherein the HERV-K-binding protein is a bispecific T cell engager (BiTE) further comprising a CD8- or CD3-binding domain.

38. The HERV-K-binding protein of claim 37, wherein the CD8-binding domain comprises SEQ ID NO: 36, or the CD3-binding domain comprises SEQ ID NO: 38.

39. An isolated polynucleotide or polynucleotides encoding the HERV-K-binding protein of claim 29.

40. A vector or vectors comprising the polynucleotide(s) of claim 39.

41. A host cell comprising the vector(s) of claim 40.

42. A method of making a HERV-K-binding protein, comprising
culturing the host cell of claim 41 under conditions that allow expression of the HERV-K-binding protein, and
optionally isolating the HERV-K-binding protein from the culture.

43. A method of treating a HERV-K positive cancer in a subject in need thereof, comprising administering to the subject a therapeutically effective amount of the HERV-K-binding protein of any one of claims 29.

44. The method of claim 43, further comprising administering to the patient an immune checkpoint inhibitor.

45. A method of treating a HERV-K positive cancer in a subject in need thereof, comprising administering to the subject a therapeutically effective amount of the T cell expressing the HERV-K-binding protein of claim 36.

46. The method of claim 45, further comprising administering to the patient an immune checkpoint inhibitor.
